(19) United States
(12) Patent Application Publication
(10) Pub. No.: US 2021/0369813 A9
(43) Pub. Date: Dec. 2, 2021

(54) MODULATING RESPONSES TO CHECKPOINT INHIBITOR THERAPY

(71) Applicants: ONCOSEC MEDICAL INCORPORATED, PENNINGTON, NJ (US); THE REGENTS OF THE UNIVERSITY OF CALIFORNIA, OAKLAND, CA (US)

(72) Inventors: ROBERT H. PIERCE, SEATTLE, WA (US); ADIL DAUD, HILLSBOROUGH, CA (US)

(21) Appl. No.: 17/140,299

(22) Filed: Jan. 4, 2021

Related U.S. Application Data
(63) Continuation of application No. 16/335,913, filed on Mar. 22, 2019.
(60) Provisional application No. 62/399,172, filed on Sep. 23, 2016.

(51) Int. Cl. A61K 38/20; A61N 1/32; A61K 39/395; C07K 16/28; A61K 41/00; A61P 35/00; A61K 9/00

(52) U.S. Cl. CPC A61K 38/20; A61N 1/327; A61K 39/39558; C07K 16/2827; A61K 41/0047; A61K 2039/876; A61P 35/00; A61K 9/0019; C07K 16/2818; A61K 9/0009; A61K 38/208

(57) ABSTRACT

The present invention provides for a dosing schedule for the intratumoral delivery of an immunostimulatory cytokine in combination with systemic delivery of a checkpoint inhibitor. In particular, it provides delivery of a plasmid encoding the immunostimulatory cytokine, e.g., IL-12, using intratumoral electroporation, and the systemic delivery of a PD-1 antagonist.

### CROSS-REFERENCE TO RELATED APPLICATIONS

The application is a continuation of U.S. application Ser. No. 16/335,913, filed Mar. 22, 2019, which was the National Stage of International Application PCT/US17/53037, filed Sep. 22, 2017, which claims the benefit of U.S. Provisional Application No. 62/399,172, filed Sep. 23, 2016, each of which is herein incorporated by reference in its entirety for all purposes.

### FIELD OF THE INVENTION

The present invention provides a method of treating a tumor by improving an immune response to a checkpoint inhibitor. In particular, an immunostimulatory cytokine is administered intratumorally to increase tumor infiltrating lymphocytes (TILs) in the tumor microenvironment.

### BACKGROUND OF THE INVENTION

Solid tumors are made up of a variety of components, including malignant cells and endothelial, structural and immune cells. Cancer cells are able to shape the microenvironment to evade immune-surveillance by the body, via “cancer immunoediting”. Tumor infiltrating lymphocytes (TILs) are frequently found in tumors, suggesting that tumors trigger an immune response in the host. This so-called tumor immunogenicity is mediated by tumor antigens. These antigens distinguish the tumor from healthy cells, thereby providing an immunological stimulus (Boon, et al. (1997) Immunol Today 18:267-268).

The concept of ‘cancer immunoediting’ describes how the immune system and tumor cells interact during the course of cancer development. It consists of three distinct phases, termed ‘the three E's’ (Kim et al. (2007) Immunology 121:1-14). Elimination entails the complete obliteration of tumor cells by T lymphocytes. In equilibrium, a population of immune-resistant tumor cells appears. Simultaneously, there is an unremitting immunological pressure on nonresistant tumor cells. This phase can last for years (Kim, et al.). Finally, during escape, the tumor has developed strategies to evade immune detection or destruction. These may be loss of tumor antigens, secretion of inhibitory cytokines, or downregulation of major histocompatibility complex molecules (Stewart and Abrams (2008) Oncogene 27:5894-5903). Additionally, antigens may be ineffectively presented to the immune system, that is, without appropriate co-stimulation, resulting in immunological tolerance (Stewart and Abrams (2008)).

Many studies report a survival benefit associated with the presence of TIL (Zhang, et al (2003) N Engl J Med 348:203-213; Sato, et al (2005) jProc Natl Acad Sci USA 102:18538-18543; Galon, et al (2006) Science 313:1960-1964; and Leffers, et al (2009) Cancer Immunol Immunother 58:449-459). This suggests that TILs are effective at delaying tumor progression, despite being antagonized by the mechanisms mentioned above.

For there to be a successful T-cell response that ultimately leads to cancer regression, three steps must occur: (1) APCs must present tumor antigen and activate an effector T-cell response (2) primed T cells must successfully home in on and infiltrate stromal tissue prior to binding to their target on the tumor, and (3) the T-cell receptors (TCRs) of the infiltrating T cells must bind to the MI-ICI-peptide complex to activate the cytotoxic T-cell response (Kelderman, et al (2014) Mol. Oncol. 8:1132-1139).

Immune checkpoint inhibitors, especially those targeting PD-1 or PD-L1, have moved to the forefront of therapeutic development in medical oncology. PD-1 on the T cell can bind to PD-Ll on the tumor cell, which sends signals to shut down the function of the T cell. In a brief period of time, a substantial amount of academic and pharmaceutical resources have refocused on developing agents targeting the PD-1/PD-L1 axis for many types of cancer. The result of these endeavors has yielded impressive clinical data, but only in a minority of patients. Often, response rates are less than 20% in unselected populations (Mahoney, et al. (2014) Oncology 28 Suppl 3:39-48).

There is evidence that T-cell homing is likely driven by the expression of certain chemokines, which are secreted by the stromal elements and tumors themselves (Gajewski et al (2010) Cancer J 16:399-403). Interleukin-12 (IL-12) is one such immunomodulatory cytokine that can increase the immune cell infiltrate in solid tumors.

Some of the anti-tumor effects of IL-12 include: increasing production of IFN-γ, which is the most potent mediator of IL-12 actions, from NK and T cells; stimulation of growth and cytotoxicity of activated NK cells, CD8+ and CD4+ T cells, shifting differentiation of CD4+ Th 0 cells toward the Th1 phenotype; enhancement of antibody-dependent cellular cytotoxicity (ADCC) against tumor cells; and the induction of IgG and suppression of IgE production from B cells. Several other mechanisms, however, also strongly contribute to antitumor activities of IL-12. These are potent antiangiogenic effects via induction of antiangiogenic cytokine and chemokine production, remodeling of the peritumoral extracellular matrix and tumor stroma, reprogramming of myeloid-derived suppressor cells, and changes in processing and increasing expression of MHC class I molecules (see, e.g., Lasek, et al. (2014) Cancer Immunol Immunother 63 :419-43 5).

However, IL-12, similar to other immunostimulatory cytokines, has proven to be too toxic for systemic administration. The present invention provides a solution to avoid systemic toxicities to IL-12, as well as increase the patient response to checkpoint inhibitors, in particular, PD-1 inhibitors.

### BRIEF DESCRIPTION OF THE DRAWING

FIG. 1 shows a schematic map of pUMVC3-hIL-12, named tavokinogene teslaplasmid (tavo) which consists of the p35 and p40 subunits of interleukin 12 (IL-12) under the control of a CMV promoter, with an internal ribosome entry site between the subunits for expression of the two subunits from a single mRNA (Aldeveron human IL-12 #4024; mouse IL-12 #4033). The mouse IL-12 plasmid construct has the mouse IL-12 subunits replacing the human IL-12 subunits

### SUMMARY OF THE INVENTION

The present invention is based, in part, upon a dosing schedule of a plasmid encoded immunostimulatory cytokine delivered intratumorally by electroporation, in combination with the systemic delivery of a checkpoint inhibitor.

The present invention provides a method of treating a cancer comprising administering to a patient a therapeutically effective amount of a checkpoint inhibitor in combination with an immunostimulatory cytokine. In some embodiments, the patient is a mammal including human, canine, feline, and equine. In further embodiments, the cancer is a melanoma. The checkpoint inhibitor can be a PD-1 antagonist including, nivolumab (ONO-4538/BMS-936558, MDX1106, OPDIVO), pembrolizumab (MK-3475, KEYTRUDA), pidilizumab (CT-011), and atezolizumab (MPDL328OA). In certain embodiments, the immunostimulatory cytokine is selected from Table 2, and in further embodiments, the immunomodulatory cytokine is IL-12. The PD-1 antagonist is delivered systemically and the immunostimulatory cytokine is encoded on a plasmid and delivered intratumorally by electroporation. It is contemplated that the PD-1 antagonist and the immunostimulatory cytokine are: a) administered together on day 1; b) the immunostimulatory cytokine is again administered on day 5 and day 8; c) the PD-1 antagonist is administered every three weeks; and d) the immunostimulatory cytokine is administered every 6 weeks. In certain embodiments, the PD-1 antagonist is selected from the group consisting of: nivolumab (ONO-4538/BMS-936558, MDX1106, OPDIVO®), pembrolizumab (MK-3475, KEYTRUDA®), pidilizumab (CT-011), and atezolizumab (MPDL328OA); and the immunostimulatory cytokine is IL-12. In further embodiments, the electroporation is at a field strength of about 200 v/cm to about 1500 V/cm, and a duration of about 100 microseconds to about 20 milliseconds. In yet another embodiment, the electroporation incorporates Electrochemical Impedance Spectroscopy (EIS).

The present invention provides a method of treating a tumor in a patient by administering a plasmid encoded immunostimulatory cytokine and a checkpoint inhibitor using a dosing schedule, wherein the dosing schedule comprises: a) a first cycle of treatment on week 1, wherein: i) the plasmid encoded immunostimulatory cytokine is delivered to a tumor by electroporation on days 1, 5, and 8; and ii) a checkpoint inhibitor delivered systemically to the patient on day 1; b) a second cycle of treatment, wherein the checkpoint inhibitor is delivered systemically to the patient three weeks after the first cycle; and c) continued subsequent treatment cycles wherein the first and second cycles are repeated alternatively every three weeks. In certain embodiments, the plasmid encoded immunostimulatory cytokine is selected from Table 2 and can be IL-12. In further embodiments, the checkpoint inhibitor is a PD-1 antagonist, including nivolumab (ONO-4538/BMS-936558, MDX1106, OPDIVO®), pembrolizumab (MK-3475, KEYTRUDA®), pidilizumab (CT-011), and atezolizumab (MPDL3280A). In an embodiment, the electroporation is at a field strength of about 200 v/cm to about 1500 V/cm, and a duration of about 100 microseconds to about 20 milliseconds. The electroporation can incorporate Electrochemical Impedance Spectroscopy (EIS). In some embodiments, the method the patient is a mammal, including human, canine, feline, and equine.

### DETAILED DESCRIPTION

As used herein, including the appended claims, the singular forms of words such as “a,” “an,” and “the,” include their corresponding plural references unless the context clearly dictates otherwise.

All references cited herein are incorporated by reference to the same extent as if each individual publication, patent application, or patent, was specifically and individually indicated to be incorporated by reference.

### I. Definitions

“Activity” of a molecule may describe or refer to the binding of the molecule to a ligand or to a receptor, to catalytic activity, to the ability to stimulate gene expression, to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” may also mean specific activity, e.g., [catalytic activity]/[mg protein], or [immunological activity]/[mg protein], or the like.

By “nucleic acid” is meant both RNA and DNA including: cDNA, genomic DNA, plasmid DNA or condensed nucleic acid, nucleic acid formulated with cationic lipids, nucleic acid formulated with peptides, cationic polymers, RNA or mRNA. In a preferred embodiment, the nucleic acid administered is a plasmid DNA which constitutes a “vector”. The nucleic acid can be, but is not limited to, a plasmid DNA vector with a eukaryotic promoter which expresses a protein with potential therapeutic action, such as, for example; IFN-α, IFN-β, IL-2, IL-12, or the like.

As used herein, “immune checkpoint” molecules refer to a group of immune cell surface receptor/ligands which induce T cell dysfunction or apoptosis. These immune inhibitory targets attenuate excessive immune reactions and ensure self-tolerance. Tumor cells harness the suppressive effects of these checkpoint molecules. Immune checkpoint target molecules include, but are not limited to, the checkpoint targets described in Table 1.

TABLE 1
Checkpoint Targets Accession Numbers

| | | GenBank | GenBank | GenBank | GenBank |
|---|---|---|---|---|---|
| | | Accession | Accession | Accession | Accession |
| | | Number- | Number- | Number- | Number- |
| | Unabbreviated | Mouse | Mouse | Human | Human |
| Target | Name | Nucleic Acid | Amino Acid | Nucleic Acid | Amino Acid |
| CTLA-4 | Cytotoxic T | U90271 | AAD00697 | L15006 | AAL07473 |
| | Lymphocyte | | | | |
| | Antigen-4 | | | | |
| PD-1 | Programmed Death 1 | NM_008798.2 | MP_032824 | NM_005018 | NP_005009.2 |
| PD-L1 | Programmed Death | GQ904197 | ADK70950 | AY254342 | AAP13470 |
| | Ligand 1 | | | | |
| LAG-3 | Lymphocyte | AY230414 | AAP57397 | X51985 | CAA36243 |
| | Activation Gene-3 | | | | |
| TIM3 | T cell | AF450241 | AAL35776 | JX049979 | AF066593 |
| | Immunoglobulin | | | | |
| | Mucin -3 | | | | |
| KIR | Killer Cell | AY130461 | AY130461.1 | AY601812 | AAT11793 |
| | Immunoglobulin-like | | | | |
| | Receptor | | | | |
| BTLA | B- and T-Lymphocyte | AY293285 | AAP44002 | AY293286 | AAP44003 |
| | Attenuator | | | | |
| A2aR | Adenosine A2a | NM_009630 | NP_033760 | NP_001265428 | NM_001278499 |
| | Receptor | | | | |
| HVEM | HerpesVirus Entry | AF515707 | AAQ08183 | AY358879 | AAQ89238 |
| | Mediator | | | | |

The phrase “immune checkpoint inhibitor” includes molecules that prevent immune suppression by blocking the effects of immune checkpoint molecules. Checkpoint inhibitors can include antibodies and antibody fragments, nanobodies, diabodies, soluble binding partners of checkpoint molecules, small molecule therapeutics, peptide antagonists, etc. Inhibitors include, but are not limited to, to the checkpoint inhibitors described in Table 1.

The phrase “immunostimulatory cytokine” includes cytokines that mediate or enhance the immune response to a foreign antigen, including viral, bacterial, or tumor antigens. Innate immunostimulatory cytokines can include, e.g., TNF-a, IL-1, IL-10, IL-12, IL-15, type I interferons (IFN-α and IFN-β), IFN-γ, and chemokines. Adaptive immunostimulatory cytokines include, e.g., IL-2, IL-4, IL-5, TGF-β, IL-10 and IFN-γ. Examples of immunostimulatory cytokines are provided in Table 2 below.

TABLE 2
Immunostimulatory Cytokines Accession Numbers

| | GenBank Accession | GenBank Accession | GenBank Accession | GenBank Accession |
|---|---|---|---|---|
| | Number-Mouse | Number-Mouse | Number-Human | Number-Human |
| Cytokine | Nucleic Acid | Amino Acid | Nucleic Acid | Amino Acid |
| TNFα | M20155 | CAA68530 | X02910 | ADV31546 |
| IL-1 | RNU48592 | CAA28637 | X03833 | CAA27448 |
| IL-10 | MUSIL1OZ | AAA39275 | HSU16720 | AAA80104 |
| IL-12 | | | | AAD16432 |
| p35 | NM_001159424.2 | NP_001152896.1 | NM_000882.3 | NP_000873.2 |
| p40 | NM_001303244.1 | NP_001290173.1 | NM_002187.2 | NP_002178.2 |
| IL-15 | NM_001254747.1 | NP_001241676 | NM_000585.4 | NP_000576 |
| IL-15Rα, | NM_008358.2 | NP_032384 | NM_002189.3 | NP_002180 |
| IFNα, | NM_010502.2 | NP_034632.2 | NM_006900.3 | NP_008831.3 |
| | | | NM_024013.2 | NP_076918.1 |
| IFNβ | NM_010510.1 | NP_034640.1 | NM_002176.3 | NP_002167.1 |
| IFNγ | NM_008337.4 | NP_032363.1 | NM_000619.2 | NP_000610.2 |
| IL-2 | NM_008366.3 | NP_032392.1 | NM_000586.3 | NP_000577.2. |
| TGFβ | NM_011577.2 | NP_035707.1 | NM_000660.5 | NP_000651.3 |

The term “cancer” includes a myriad of diseases generally characterized by inappropriate cellular proliferation, abnormal or excessive cellular proliferation. Examples of cancer include but are not limited to, breast cancer, colon cancer, prostate cancer, pancreatic cancer, melanoma, lung cancer, ovarian cancer, kidney cancer, brain cancer, or sarcomas. Such cancers may be caused by, environmental factors, chromosomal abnormalities, degenerative growth and developmental disorders, mitogenic agents, ultraviolet radiation (UV), viral infections, inappropriate tissue expression of a gene, alterations in expression of a gene, or carcinogenic agents.

The term “treatment” includes, but is not limited to, inhibition or reduction of proliferation of cancer cells, destruction of cancer cells, prevention of proliferation of cancer cells or prevention of initiation of malignant cells or arrest or reversal of the progression of transformed premalignant cells to malignant disease or amelioration of the disease.

The term “subject” refers to any animal, preferably a mammal such as a human. Veterinary uses are also intended to be encompassed by this invention, including canine and feline.

The terms “electroporation”, “electro-permeabilization,” or “electro-kinetic enhancement” (“EP”) as used interchangeably herein refer to the use of a transmembrane electric field pulse to induce microscopic pathways (pores) in a bio-membrane; their presence allows biomolecules such as plasmids, oligonucleotides, siRNA, drugs, ions, and water to pass from one side of the cellular membrane to the other.

The term “biomolecule” as used herein, encompasses plasmid encoded antibodies, antibody fragments, full length immunomodulatory proteins, soluble domains of membrane anchored molecules, fusion proteins, and the like.

The term “pUMVC3-hIL-12” as used herein encompasses plasmid encoded human IL-12, more particularly, tavokinogene teslaplasmid, hereinafter, “tavo”.

The phrase “intratumoral delivery of plasmid IL-12 by electroporation” or “IT- pIL-12-EP” is encompassed by “ImmunoPulseg IL-12” or “ImmunoPulseg mIL-12”.

### II. General

The present invention encompasses a method of treating cancer, in particular, a melanoma, and the surprising result of a dosing treatment schedule that increases the number of Tumor Infiltrating Lymphocytes (TILs) in the tumor microenvironment and improves a patient's response to checkpoint inhibitor therapy, e.g., treatment with PD-1 antagonists.

### III. Antibodies

The present invention provides an immunotherapeutic approach for reducing the size of a tumor or inhibiting the growth of cancer cells in an individual, or reducing or inhibiting the development of metastatic cancer in an individual suffering from cancer. Therapy is achieved by either systemic delivery of protein therapeutics, or intratumoral delivery of plasmids encoding various soluble forms of checkpoint inhibitors, using electroporation. Checkpoint inhibitor therapy may occur before, during, or after intratumoral delivery by electroporation of an immunostimulatory cytokine, e.g., IL-12. In particular,

Checkpoint inhibitors may be in the form of antibodies or antibody fragments, both of which can be encoded in a plasmid and delivered to the tumor by electroporation, or delivered as proteins/peptides systemically. As noted above, delivery of the checkpoint inhibitor therapeutic can occur before, during or after intratumoral delivery by electroporation of an immunostimulatory cytokine, e.g., IL-12.

The term “antibody” as used herein includes immunoglobulins, which are the product of B cells and variants thereof. An immunoglobulin is a protein comprising one or more polypeptides substantially encoded by the immunoglobulin kappa and lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Also subclasses of the heavy chain are known. For example, IgG heavy chains in humans can be any of IgG1, IgG2, IgG3 and IgG4 subclass.

A typical immunoglobulin structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains, respectively.

Antibodies exist as full-length intact antibodies or as a number of well-characterized fragments produced by digestion with various peptidases or chemicals. Thus, for example, pepsin digests an antibody below the disulfide linkages in the hinge region to produce F(ab′)2, a dimer of Fab which itself is a light chain joined to VH-CH₁ by a disulfide bond. The F(ab′)2 may be reduced under mild conditions to break the disulfide linkage in the hinge region thereby converting the F(ab′)2 dimer into a Fab' monomer. The Fab′ monomer is essentially a Fab fragment with the hinge region (see, Fundamental Immunology, W. E. Paul, ed., Raven Press, N.Y. (1993), for a more detailed description of other antibody fragments). A Fab fragment and Fc fragment are generated by digesting IgG with papain. Papain cleaves in the hinge region just above the residues involved in interchain S—S bonding, resulting in monovalent Fab fragments and the Fc fragment, which includes two constant region fragments, each containing the lower part of the hinge, CH2 and CH3 domains. The constant region fragments of the Fc are stabilized as a dimer though interchain S—S bonding of the lower residues of the hinge region.

Immunoglobulin “Fc” classically refers to the portion of the constant region generated by digestion with papain. Includes the lower hinge which has the interchain S—S bonds. The term “Fc” as used herein refers to a dimeric protein comprising a pair of immunoglobulin constant region polypeptides, each containing the lower part of the hinge, CH2 and CH3 domain. Such “Fc” fragment may or may not contain S—S interchain bridging in the hinge region. It should be understood that an Fc may be from any Ig class and, as such, may include a CH4 domain such as in the case of IgM. Mutant sequences of an Fc are known such as described by Wines et al., J. Immunol. 2000 May 15; 164(10):5313-8 and may be used herein.

While various antibody fragments are defined in terms of the digestion of an intact antibody, one of skill will appreciate that any of a variety of antibody fragments may be synthesized de novo either chemically or by utilizing recombinant DNA methodology. Thus, the term antibody, as used herein also includes antibody fragments either produced by the modification of whole antibodies or synthesized de novo or antibodies and fragments obtained by using recombinant DNA methodologies.

Recombinant antibodies may be conventional full length antibodies, antibody fragments known from proteolytic digestion, unique antibody fragments such as Fv or single chain Fv (scFv), domain deleted antibodies, and the like. Fragments may include domains or polypeptides with as little as one or a few amino acid deleted or mutated while more extensive deletion is possible such as deletion of one or more domains.

An Fv antibody is about 50 kD in size and comprises the variable regions of the light and heavy chain. A single chain Fv (“scFv”) polypeptide is a covalently linked VH:VL heterodimer which may be expressed from a nucleic acid including VH- and VL-encoding sequences either joined directly or joined by a peptide-encoding linker. See e.g., Huston, et al. (1988) Proc. Nat. Acad. Sci. USA, 85:5879-5883. A number of structures for converting the naturally aggregated, but chemically separated light and heavy polypeptide chains from an antibody V region into an scFv molecule which will fold into a three-dimensional structure substantially similar to the structure of an antigen-binding site.

An alternative to the traditional antibody fragments above has been found in a set of unique antibodies produced by the immune systems of camels, llamas, and sharks. Unlike other antibodies, these affinity reagents are composed of only two heavy chains; better yet, a single domain forms the antigen-binding sites for these heavy-chain antibodies. The domains can even be genetically engineered to produce extremely small, very stable single-domain recombinant antibody fragments, called “nanobodies.” Plasmids encoding heavy chain only (VHH), single domain antibodies, and nanobodies are also contemplated for intratumoral delivery by electroporation.

### IV. Soluble Antagonists

Antagonists/inhibitors of checkpoint molecules may also be soluble binding partners of the checkpoint inhibitors, such as soluble PD-L1, which comprises at least the extracellular domain (ECD) of PD-L 1. Other soluble checkpoint inhibitors will similarly lack transmembrane and intracellular domains, but are capable of binding to their binding partners and eliciting a biological effect. For intratumoral delivery by electroporation, the ECDs will be encoded in an expression vector and will be expressed when delivered to the tumor.

The soluble encoded form of the checkpoint inhibitor may be linked in the expression vector to DNA encoding another protein or polypeptide. Such other polypeptide may be the Fc portion of an immunoglobulin, albumin, or any other type of serum protein or fragment thereof which maintains the solubility of the checkpoint inhibitor molecule. The soluble form of the checkpoint inhibitor molecule may be linked to an immunoglobulin via the heavy and/or light chain, which may be a fragment or a full length heavy or light chain. The immunoglobulin may be an antibody that can target an antigen associated with a cancer cell or tumor.

The soluble checkpoint inhibitor is delivered either as protein systemically or intratumorally via electroporation, as a nucleic acid. Nucleic acid refers to a polynucleotide compound, which includes oligonucleotides, comprising nucleosides or nucleoside analogs that have nitrogenous heterocyclic bases or base analogs, covalently linked by standard phosphodiester bonds or other linkages. Nucleic acids can include RNA, DNA, chimeric DNA-RNA polymers, or analogs thereof. The DNA can be a plasmid expressing a particular soluble checkpoint inhibitor molecule of interest.

V. Expression plasmids

As used herein, the term a “plasmid” refers to a construct made up of genetic material (i.e., nucleic acids). The DNA plasmid is one that includes an encoding sequence of a recombinant polypeptide that is capable of being expressed in a mammalian cell, upon said DNA plasmid entering after electroporation. Preferably, the encoding sequence is an immunostimulatory cytokine that elicits an immune response in the target mammal, specifically in a tumor. In some embodiments, the encoding sequence is in constructs optimized for mammalian expression, which can include one or more of the following: including the addition of a Kozak sequence, codon optimization, RNA optimization, and integration of translation modifiers, such as IRES or P2A sequences.

It includes genetic elements arranged such that an inserted coding sequence can be transcribed in eukaryotic cells. Also, while the plasmid may include a sequence from a viral nucleic acid, such viral sequence preferably does not cause the incorporation of the plasmid into a viral particle, and the plasmid is therefore a non-viral vector. Preferably, a plasmid is a closed circular DNA molecule. The enhancer/promoter region of an expression plasmid will determine the levels of expression. Most of the gene expression systems designed for high levels of expression contain the intact human cytomegalovirus (CMV) immediate early enhancer/promoter sequence. However, down-regulation of the CMV promoter over time has been reported in tissues. The hypermethylation of the CMV promoter, as observed when incorporated into retroviral vectors, has not been observed for episomal plasmids in vivo. Nevertheless, the CMV promoter silencing could be linked to its sensitivity to reduced levels of the transcription factor NF-κB. The activity of the CMV promoter has also been shown to be attenuated by various cytokines including interferons (α and β), and tumor necrosis factor (TNF-α). In order to prolong expression in vivo and ensure specificity of expression in desired tissues, tissue-specific enhancer/promoters have been incorporated in expression plasmids. The chicken skeletal alpha actin promoter has been shown to provide high levels of expression (equivalent to the ones achieved with a CMV-driven construct) for several weeks in non-avian striated muscles.

Additional genetic sequences in the expression plasmids can be added to influence the stability of the messenger RNA (mRNA) and the efficiency of translation. The 5′ untranslated region (5′ UTR) is known to effect translation and it is located between the cap site and the initiation codon. The 5′ UTR should ideally be relatively short, devoid of strong secondary structure and upstream initiation codons, and should have an initiation codon AUG within an optimal local context. The 5′ UTR can also influence RNA stability, RNA processing and transcription. In order to maximize gene expression by ensuring effective and accurate RNA splicing, one or more introns can be included in the expression plasmids at specific locations. The possibility of inefficient and/or inaccurate splicing can be minimized by using synthetic introns that have idealized splice junction and branch point sequences that match the consensus sequence. Another important sequence within a gene expression system is the 3′ untranslated region (3′ UTR), a sequence in the mRNA that extends from the stop codon to the poly(A) addition site. The 3′ UTR can influence mRNA stability, translation and intracellular localization. The skeletal muscle α-actin 3′ UTR has been shown to stabilize mRNA in muscle tissues thus leading to higher levels of expression as compared to other 3′ UTR. This 3′ UTR appears to induce a different intracellular compartmentalization of the produced proteins, preventing the effective trafficking of the proteins to the secretory pathway and favoring their perinuclear localization.

In some embodiments, the DNA plasmid can be manufactured, preferably in large scale quantities, using a process that is enhanced for high yield and/or cGMP manufacturing. Preferably, the DNA plasmid that is manufactured for delivery to mammals can be formulated into high DNA concentrations. The DNA plasmid manufacturing process can be performed by transfecting microbial cells, such as E. coli cells. The processes contemplated for manufacturing DNA plasmids described herein include that disclosed in U.S. Pat. No. 7,238,522), which is hereby incorporated in their entirety. The DNA plasmids are preferably formulated to be safe and effective for injection into mammal subjects. Preferably, the DNA plasmids are formulated to be in concentrations sufficient to be expressed by the transformed cell.

### VI. Disorders.

The present invention is contemplated for treating patients afflicted with cancer or other non-cancerous (benign) growths. These growths may manifest themselves as any of a lesion, polyp, neoplasm (e.g. papillary urothelial neoplasm), papilloma, malignancy, tumor (e.g. Klatskin tumor, hilar tumor, noninvasive papillary urothelial tumor, germ cell tumor, Ewing's tumor, Askin's tumor, primitive neuroectodermal tumor, Leydig cell tumor, Wilms' tumor, Sertoli cell tumor), sarcoma, carcinoma (e.g. squamous cell carcinoma, cloacogenic carcinoma, adenocarcinoma, adenosquamous carcinoma, cholangiocarcinoma, hepatocellular carcinoma, invasive papillary urothelial carcinoma, flat urothelial carcinoma), lump, or any other type of cancerous or non-cancerous growth. Tumors treated with the methods of the present embodiment may be any of noninvasive, invasive, superficial, papillary, flat, metastatic, localized, unicentric, multicentric, low grade, and high grade.

The present invention is intended for the treatment of numerous types of malignant tumors (i.e. cancer) and benign tumors. For example, adrenal cortical cancer, anal cancer, bile duct cancer (e.g. periphilar cancer, distal bile duct cancer, intrahepatic bile duct cancer) bladder cancer, benign and cancerous bone cancer (e.g. osteoma, osteoid osteoma, osteoblastoma, osteochrondroma, hemangioma, chondromyxoid fibroma, osteosarcoma, chondrosarcoma, fibrosarcoma, malignant fibrous histiocytoma, giant cell tumor of the bone, chordoma, lymphoma, multiple myeloma), brain and central nervous system cancer (e.g. meningioma, astocytoma, oligodendrogliomas, ependymoma, gliomas, medulloblastoma, ganglioglioma, Schwannoma, germinoma, craniopharyngioma), breast cancer (e.g. ductal carcinoma in situ, infiltrating ductal carcinoma, infiltrating lobular carcinoma, lobular carcinoma in situ, gynecomastia), Castleman disease (e.g. giant lymph node hyperplasia, angiofollicular lymph node hyperplasia), cervical cancer, colorectal cancer, endometrial cancer (e.g. endometrial adenocarcinoma, adenocanthoma, papillary serous adnocarcinoma, clear cell) esophagus cancer, gallbladder cancer (mucinous adenocarcinoma, small cell carcinoma), gastrointestinal carcinoid tumors (e.g. choriocarcinoma, chorioadenoma destruens), Hodgkin's disease, non-Hodgkin's lymphoma, Cutaneous T-Cell Lymphoma (CTCL), Kaposi's sarcoma, kidney cancer (e.g. renal cell cancer), laryngeal and hypopharyngeal cancer, liver cancer (e.g. hemangioma, hepatic adenoma, focal nodular hyperplasia, hepatocellular carcinoma), lung cancer (e.g. small cell lung cancer, non-small cell lung cancer), mesothelioma, plasmacytoma, nasal cavity and paranasal sinus cancer (e.g. esthesioneuroblastoma, midline granuloma), nasopharyngeal cancer, head and neck squamous cell carcinoma, neuroblastoma, oral cavity and oropharyngeal cancer, ovarian cancer, pancreatic cancer, penile cancer, pituitary cancer, prostate cancer, retinoblastoma, rhabdomyosarcoma (e.g. embryonal rhabdomyosarcoma, alveolar rhabdomyosarcoma, pleomorphic rhabdomyosarcoma), salivary gland cancer, skin cancer, both melanoma, in particular, metastatic melanoma, and non-melanoma skin cancer (including Merkel Cell Carcinoma), stomach cancer, testicular cancer (e.g. seminoma, nonseminoma germ cell cancer), thymus cancer, thyroid cancer (e.g. follicular carcinoma, anaplastic carcinoma, poorly differentiated carcinoma, medullary thyroid carcinoma, thyroid lymphoma), vaginal cancer, vulvar cancer, and uterine cancer (e.g. uterine leiomyosarcoma).

### VII. Electroporation; Devices

The invention finds use in intratumoral gene electrotransfer. In particular, the current plasmid constructs can be used to generate adequate concentrations of several recombinantly expressed immunomodulatory molecules such as, multimeric cytokines or combination of multimeric cytokines, co-stimulatory molecules in native or engineered forms, genetic adjuvants containing shared tumor antigens, etc. To achieve transfer of the instant plasmid constructs into a tissue, e.g., a tumor, an electroporation device is employed.

The devices and methods of the present embodiment work to treat cancerous tumors by delivering electrical therapy continuously and/or in pulses for a period of time ranging from a fraction of a second to several days, weeks, and/or months to tumors. In a preferred embodiment, electrical therapy is direct current electrical therapy.

The term “electroporation” (i.e. rendering cellular membranes permeable) as used herein may be caused by any amount of coulombs, voltage, and/or current delivered to a patient in any period of time sufficient to open holes in cellular membranes (e.g. to allow diffusion of molecules such as pharmaceuticals, solutions, genes, and other agents into a viable cell).

Delivering electrical therapy to tissue causes a series of biological and electrochemical reactions. At a high enough voltage, cellular structures and cellular metabolism are severely disturbed by the application of electrical therapy. Although both cancerous and non-cancerous cells are destroyed at certain levels of electrical therapy tumor cells are more sensitive to changes in their microenvironment than are non-cancerous cells. Distributions of macroelements and microelements are changed as a result of electrical therapy. Destruction of cells in the vicinity of the electroporation is known as irreversible electroporation.

The use of reversible electroporation is also contemplated. Reversible electroporation occurs when the electricity applied with the electrodes is below the electric field threshold of the target tissue. Because the electricity applied is below the cells' threshold, cells are able to repair their phospholipid bilayer and continue on with their normal cell functions. Reversible electroporation is typically done with treatments that involve getting a drug or gene (or other molecule that is not normally permeable to the cell membrane) into the cell. (Garcia, et al. (2010) “Non-thermal irreversible electroporation for deep intracranial disorders”. 2010 Annual International Conference of the IEEE Engineering in Medicine and Biology: 2743-6.)

In a single electrode configuration, voltage may be applied for fractions of seconds to hours between a lead electrode and the generator housing, to begin destruction of cancerous tissue. Application of a given voltage may be in a series of pulses, with each pulse lasting fractions of a second to several minutes. In certain embodiments, the pulse duration or width can be from about. Low voltage may also be applied for of a duration of fractions of seconds to minutes, which may attract white blood cells to the tumor site. In this way, the cell mediated immune system may remove dead tumor cells and may develop antibodies against tumor cells. Furthermore, the stimulated immune system may attack borderline tumor cells and metastases.

Various adjuvants may be used to increase any immunological response, depending on the host species, including but not limited to Freund's adjuvant (complete and incomplete), mineral salts such as aluminum hydroxide or aluminum phosphate, various cytokines, surface active substances such as lysolecithin, pluronic polyols, polyanions, peptides, oil emulsions, and potentially useful human adjuvants such as BCG (bacille Calmette-Guerin) and Corynebacterium parvum. Alternatively, the immune response could be enhanced by combination and or coupling with molecules such as keyhole limpet hemocyanin, tetanus toxoid, diptheria toxoid, ovalbumin, cholera toxin or fragments thereof.

U.S. Pat. No. 7,245,963 by Draghia-Akli, et al. describes modular electrode systems and their use for facilitating the introduction of a biomolecule into cells of a selected tissue in a body or plant. The modular electrode systems comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The biomolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the biomolecule into the cell between the plurality of electrodes. The entire content of U.S. Pat. No. 7,245,963 is hereby incorporated by reference.

U.S. Patent Pub. 2005/0052630 describes an electroporation device which may be used to effectively facilitate the introduction of a biomolecule into cells of a selected tissue in a body or plant. The electroporation device comprises an electro-kinetic device (“EKD device”) whose operation is specified by software or firmware. The EKD device produces a series of programmable constant-current pulse patterns between electrodes in an array based on user control and input of the pulse parameters, and allows the storage and acquisition of current waveform data. The electroporation device also comprises a replaceable electrode disk having an array of needle electrodes, a central injection channel for an injection needle, and a removable guide disk (see, e.g., U.S. Patent Pub. 2005/0052630) is hereby incorporated by reference.

The electrode arrays and methods described in U.S. Pat. No. 7,245,963 and U.S. Patent Pub. 2005/0052630 are adapted for deep penetration into not only tissues such as muscle, but also other tissues or organs. Because of the configuration of the electrode array, the injection needle (to deliver the biomolecule of choice) is also inserted completely into the target organ, and the injection is administered perpendicular to the target issue, in the area that is pre-delineated by the electrodes.

Efficiency of uptake using electroporation is dependent on a variety of interrelated factors including but not limited to the nature of the tissue, waveform of the electrical signal, the nature of the electric field, pulse length. The various parameters including electric field strengths required for the electroporation of any known cell are generally described in the scientific literature.

The nature of the electric field to be generated is determined by the nature of the tissue, the size of the selected tissue and its location. It is desirable that the field be as homogenous as possible and of the correct amplitude. Excessive field strength results in lysing of cells, whereas a low field strength results in reduced efficacy. Typically, the electric fields needed for in vivo cell electroporation are generally similar in magnitude to the fields required for cells in vitro. In one embodiment, the magnitude of the electric field range from approximately, 10 V/cm to about 1500 V/cm, preferably from about 700 V/cm to 1500 V/cm and preferably from about 1000 V/cm to 1500 V/cm. When lower field strengths (from about 10 V/cm to 100 V/cm, and more preferably from about 25 V/cm to 75 V/cm) are employed, the pulse length is long. For example, when the nominal electric field is about 25-75 V/cm, it is preferred that the pulse length is about 10 msec.

The waveform of the electrical signal provided by the pulse generator can be an exponentially decaying pulse, a square pulse, a unipolar oscillating pulse train, a bipolar oscillating pulse train, or a combination of any of these forms. The square wave electroporation pulses have a gentler effect on the cells which results in higher cell viability. Square wave electroporation systems deliver controlled electric pulses that rise quickly to a set voltage, stay at that level for a set length of time (pulse length), and then quickly drop to zero. This type of system yields better transformation efficiency for the electroporation of plant protoplast and mammalian cell lines than an exponential decay system.

The pulse length can be about 10 μs to about 100 ms. There can be any desired number of pulses, typically one to 100 pulses per second. The interval between pulses sets can be any desired time, such as one second. The waveform, electric field strength and pulse duration may also depend upon the type of cells and the type of molecules that are to enter the cells via electroporation.

Other alternative electroporation technologies are also contemplated. In vivo plasmid delivery can also be performed using cold plasma. Plasma is one of the four fundamental states of matter, the others being solid, liquid, and gas. Plasma is an electrically neutral medium of unbound positive and negative particles (i.e. the overall charge of a plasma is roughly zero). A plasma can be created by heating a gas or subjecting it to a strong electromagnetic field, applied with a laser or microwave generator. This decreases or increases the number of electrons, creating positive or negative charged particles called ions (Luo, et al. (1998) Phys. Plasma 5:2868-2870) and is accompanied by the dissociation of molecular bonds, if present.

To maximize the efficacy of EP, a quantifiable metric of membrane integrity that is measurable in real-time is desirable. Electrochemical impedance spectroscopy (EIS) is a method for the characterization of physiologic and chemical systems and can be performed with standard EP electrodes. This technique measures the electrical response of a system over a range of frequencies to reveal energy storage and dissipation properties. In biologic systems the extracellular and intracellular matrix resist current flow and therefore can be electrically represented as resistors. The lipids of intact cell membranes and organelles store energy and are represented as capacitors. Electrical impedance is the sum of these resistive and capacitive elements over a range of frequencies. To quantify each of these parameters, tissue impedance data can be fit to an equivalent circuit model. Real-time monitoring of electrical properties of tissues will enable feedback control over EP parameters and lead to optimum transfection in heterogeneous tumors. Using EIS feedback, will allow (1) delivery parameters to be adjusted in real-time, (2) delivery of only the pulses necessary to generate a therapeutic response, and (3) reduce the overall EP-mediated tissue damage as a result. successful EP occurs when the cellular membrane is disrupted, resulting in a change of capacitance. Thus, by monitoring and measuring electrical properties, e.g. impedance (including capacitance) before, during and/or after the EP pulses, relevant empirical data can be collected and used to create models during initial training phases. A full description of EIS EP can be found in PCT/US16/25416, which is incorporated by reference and attached as Appendix A.

Other alternative electroporation technologies are also contemplated. In vivo plasmid delivery can also be performed using cold plasma. Plasma is one of the four fundamental states of matter, the others being solid, liquid, and gas. Plasma is an electrically neutral medium of unbound positive and negative particles (i.e. the overall charge of a plasma is roughly zero). A plasma can be created by heating a gas or subjecting it to a strong electromagnetic field, applied with a laser or microwave generator. This decreases or increases the number of electrons, creating positive or negative charged particles called ions (Luo, et al. (1998)Phys. Plasma 5:2868-2870) and is accompanied by the dissociation of molecular bonds, if present.

Cold plasmas (i.e., non-thermal plasmas) are produced by the delivery of pulsed high voltage signals to a suitable electrode. Cold plasma devices may take the form of a gas jet device or a dielectric barrier discharge (DBD) device. Cold temperature plasmas have attracted a great deal of enthusiasm and interest by virtue of their provision of plasmas at relatively low gas temperatures. The provision of plasmas at such a temperature is of interest to a variety of applications, including wound healing, anti-bacterial processes, various other medical therapies and sterilization. As noted earlier, cold plasmas (i.e., non-thermal plasmas) are produced by the delivery of pulsed high voltage signals to a suitable electrode. Cold plasma devices may take the form of a gas jet device, a dielectric barrier discharge (DBD) device or multi-frequency harmonic-rich power supply.

Dielectric barrier discharge device, relies on a different process to generate the cold plasma. A dielectric barrier discharge (DBD) device contains at least one conductive electrode covered by a dielectric layer. The electrical return path is formed by the ground that can be provided by the target substrate undergoing the cold plasma treatment or by providing an in-built ground for the electrode. Energy for the dielectric barrier discharge device can be provided by a high voltage power supply, such as that mentioned above. More generally, energy is input to the dielectric barrier discharge device in the form of pulsed DC electrical voltage to form the plasma discharge. By virtue of the dielectric layer, the discharge is separated from the conductive electrode and electrode etching and gas heating is reduced. The pulsed DC electrical voltage can be varied in amplitude and frequency to achieve varying regimes of operation. Any device incorporating such a principle of cold plasma generation (e.g., a DBD electrode device) falls within the scope of various embodiments of the present invention.

Cold plasma has been employed to transfect cells with foreign nucleic acids. In particular, transfection of tumor cells (see, e.g., Connolly, et al. (2012) Human Vaccines & Immunotherapeutics 8:1729-1733; and Connolly et al (2015) Bioelectrochemistry 103: 15-21).

### VIII. Dosing Schedules

The present invention describes a dosing regimen encompassing administration of a plasmid encoded immunostimulatory cytokine by electroporation, in combination with a checkpoint inhibitor for a number of cycles. It may be desirable to administer the two therapies concurrently, sequentially, or separately. In some embodiments, the plasmid encoded immune stimulatory cytokine is administered at every cycle or alternate cycles. In further embodiments, the plasmid encoded immunostimulatory cytokine and the checkpoint inhibitor can be delivered concurrently on Day 1 of each cycle. In preferred embodiments the two therapies are administered concurrently on odd numbered cycles and the checkpoint inhibitor is administered alone on even numbered cycles.

The plasmid encoded immunostimulatory cytokine is delivered by electroporation at least one, two, or three days of each cycle or alternating cycles. In certain embodiments, the cytokine is delivered on days 1, 5, and 8 of each cycle. In a preferred embodiment, the cytokine is delivered on days 1, 3, and 8 of every odd numbered cycle.

The intervening period between each cycle can be from about 1 week to about 6 weeks, from about 2 weeks to about 5 weeks. In a preferred embodiment, the intervening period between cycles is about 3 weeks.

When PD-1 antagonists, specifically pembrolizumab, are used, the systemic dosing is between 2 mg/kg -10 mg/kg, preferably 2 mg/kg. Alternatively dosing of pembrolizumab is between 100-500 mg per cycle, preferably 200-400 mg per cycle, and most preferably 200 mg per cycle.

The broad scope of this invention is best understood with reference to the following examples, which are not intended to limit the inventions to the specific embodiments.

### EXAMPLES

### General Methods.

Standard methods in molecular biology are described. Maniatis et al. (1982) Molecular Cloning, A Laboratory Manual, Cold Spring Harbor Laboratory Press, Cold Spring Harbor, N.Y.; Sambrook and Russell (2001) Molecular Cloning, 3rd ed., Cold Spring Harbor Laboratory Press, Cold Spring Harbor, N.Y.; Wu (1993) Recombinant DNA, Vol. 217, Academic Press, San Diego, Calif. Standard methods also appear in Ausbel et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described. Coligan et al. (2000) Current Protocols in Protein Science, Vol. 1, John Wiley and Sons, Inc., New York. Chemical analysis, chemical modification, post-translational modification, production of fusion proteins, glycosylation of proteins are described. See, e.g., Coligan et al. (2000) Current Protocols in Protein Science, Vol. 2, John Wiley and Sons, Inc., New York; Ausubel et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391. Production, purification, and fragmentation of polyclonal and monoclonal antibodies are described. Coligan et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra. Standard techniques for characterizing ligand/receptor interactions are available. See, e.g., Coligan et al. (2001) Current Protocols in Immunology, Vol. 4, John Wiley, Inc., New York.

Methods for flow cytometry, including fluorescence activated cell sorting detection systems (FACS®), are available. See, e.g., Owens et al. (1994) Flow Cytometry Principles for Clinical Laboratory Practice, John Wiley and Sons, Hoboken, N.J.; Givan (2001) Flow Cytometry, 2nd ed.; Wiley-Liss, Hoboken, N.J.; Shapiro (2003) Practical Flow Cytometry, John Wiley and Sons, Hoboken, N.J. Fluorescent reagents suitable for modifying nucleic acids, including nucleic acid primers and probes, polypeptides, and antibodies, for use, e.g., as diagnostic reagents, are available. Molecular Probes (2003) Catalogue, Molecular Probes, Inc., Eugene, Oreg.; Sigma-Aldrich (2003) Catalogue, St. Louis, Mo.

Standard methods of histology of the immune system are described. See, e.g., Muller-Harmelink (ed.) (1986) an Thymus: Histopathology and Pathologypringer Verlag, New York, N.Y.; Hiatt, et al. (2000) or Atlas of Histology, Lippincott, Williams, and Wilkins, Phila, Pa.; Louis, et al. (2002) Basic Histology: Text and Atlas, McGraw-Hill, New York, N.Y.

Software packages and databases for determining, e.g., antigenic fragments, leader sequences, protein folding, functional domains, glycosylation sites, and sequence alignments, are available. See, e.g., GenBank, Vector NTI® Suite (Informax, Inc., Bethesda, Md.); GCG Wisconsin Package (Accelrys, Inc., San Diego, Calif.); DeCypher® (TimeLogic Corp., Crystal Bay, Nev.); Menne et al. (2000) Bioinformatics 16: 741-742; Menne et al. (2000) Bioinformatics Applications Note 16:741-742; Wren et al. (2002) Comput. Methods Programs Biomed. 68:177-181; von Heijne (1983) Eur. J. Biochem. 133:17-21; von Heijne (1986) Nucleic Acids Res. 14:4683-4690.

### II. Preclinical

### A. Syngeneic Mouse Tumor Models

Female C57B1/6J mice, 6-8 weeks of age were obtained from Jackson Laboratories and housed in accordance with AALAM guidelines.

B16.F10 or B160VA cells were cultured with McCoy's 5A medium (2 mM L-Glutamine) supplemented with 10% FBS and 50 μg/mL gentamicin. Cells were harvested by digestion with 0.25% trypsin and re-suspended in Hank's balanced salt solution (HBSS). Anesthetized mice were subcutaneously injected with 1 million cells in a total volume of 0.1 ml into the right flank of each mouse. 0.5 million cells in a total volume of 0.1 ml were injected subcutaneously into the left flank of each mouse Tumor growth was monitored by digital caliper measurements starting day 8 until average tumor volume reaches ˜100 mm³. Once tumors are staged to the desired volume, mice with very large or small tumors were culled. Remaining mice were divided into groups of 10 mice each, randomized by tumor volume implanted on right flank.

This protocol was used as a standard model to test simultaneously for the effect on the treated tumor (primary) and untreated (contralateral). Tumor volumes were measured twice weekly. Mice were euthanized when the total tumor burden of the primary and contralateral reached 2000 mm³.

### B. Intratumoral Electroporation

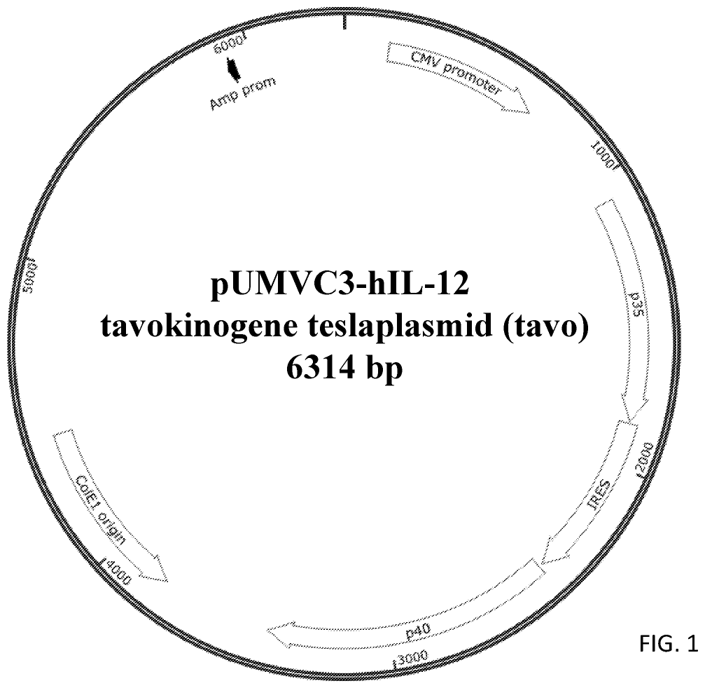

Mice were anesthetized with isoflurane for treatment. Circular plasmid DNA was diluted to 1 μg/μL in sterile 0.9% saline. 50 μL of plasmid DNA encoding mouse IL-12 was injected centrally into primary tumors using a 1 ml syringe with a 26 Ga needle. The plasmid structure for expression of mouse 11-12 is the same as that for human IL-12 (tavo) shown in FIG. 1. In some experiments, a different version of this plasmid was used with the exon skipping motif, P2A substituted for the internal ribosome entry site (IRES). In some experiments, the empty pUMVC3 vector was injected as a control. Electroporation was performed immediately after injection. Electroporation of DNA was achieved using a MedPulser with clinical electroporation parameters of 1500 V/cm, 100 μs pulses, 0.5 cm, 6-needle electrode. Alternative parameters used were 350 V/cm, 10-msec pulses, 300 ms pulse frequency, 0.5 cm acupuncture needles. This procedure is referred to hereafter as ImmunoPulse® mIL-12.

TABLE 3
B16F0 tumor regression for treated and untreated tumors after intratumoral
electroporation. Electroporation with the parameters of 1500 V/cm, 100 μs,
0.5 cm, 6 needle electrode was performed 8, 12, and 15 days after
tumor cell implantation. Tumor volume measurements shown were
taken on Day 16.

| | Tumor volume (mm³), | |
|---|---|---|
| | Mean ± SEM, n = 10 | |
| Treatment | Treated tumor | Untreated tumor |
| Untreated | 1005.2 ± 107.4 | 626.6 ± 71.8 |
| ImmunoPulse ® pUMVC3 control | 345.2 ± 130.5 | 951.1 ± 77.0 |
| ImmunoPulse ® mIL-12 | 140.3 ± 49.8 | 441.0 ± 80.8 |

Intratumoral electroporation of a plasmid encoding mouse IL-12 (ImmunoPulse® mIL-12) caused significantly reduced tumor growth of both treated and untreated, contralateral tumors.

### C. Intraperitoneal Injection

Injection solution were brought up to room temperature and drawn up in a syringe affixed with a sterile 27-guage needle. The injection site was disinfected with an alcohol-soaked pad. 200 μL of antibody solution was injected into the right side just above the midline at a 30-degree angle to a depth of 0.5 cm. Mice were treated twice a week with 200 μL of a 1 mg/mL control IgG (Clone: LTF-2, BioXCell catalog#: BE0090) or Anti-PD-L 1 (Clone: 10F.9G2; BioXCell catalog#: BE0101) solution.

TABLE 4
Tumor regression in a contralateral B16F10 tumor model treated
with ImmunoPulse ® mIL-12 and anti-PDL1 antibody. ImmunoPulse ®
mIL-12 was performed with 350 V/cm, 10-msec pulses on Day 0
and Day 7 (8 and 15 days after tumor cell implantation). Anti-PD-Ll
antibody was injected IP twice weekly starting on Day 0. Day 7.
Tumor volume measurements are shown for Day 16.

| | Tumor volume (mm³), | |
|---|---|---|
| | Mean +/− SEM, n = 9 | |
| Treatment | Treated tumor | Untreated tumor |
| ImmunoPulse ® mIL-12 + IgG control | 324.4 +/− 145.2 | 509.7 +/− 154.5 |
| ImmunoPulse ® mIL-12 + aPD-L1 | 39.1 +/− 26.7 | 165.9 +/− 41.5 |

Additional treatment with antagonistic anti-PD-L1 antibody improved regression of both ImmunoPulse® mIL-12 treated and untreated Bl6F10 tumors in mice, suggesting a positive effect of combining intratumoral IL-12 gene therapy and systemic inhibition of the PD-1/PD-L1 signaling pathway.

D. Flow cytometry

At various time points after ImmunoPulse® mIL-12 treatment, mice were sacrificed and tumor and spleen tissue were surgically removed.

Splenocytes were isolated by pressing spleens through a 70 micron filter, followed by red blood cell lysis (RBC lysis buffer, VWR, 4203010BL), and lympholyte (Cedarlane CL5035) fractionation. Lymphocytes were stained with SIINFEKL-tetramers (MBL International T03002), followed by staining with antibody cocktails containing: anti-CD3 (Biolegend 100225), anti-CD4 (Biolegend 100451), anti-CD8a (Biolegend 100742), anti-CD19 (Biolegend 115546), and vital stain (live-dead Aqua; Thermo-Fisher L-34966). Cells were fixed and analyzed on an LSR II flow cytometer (Beckman).

Tumors were dissociated using Gentle-MACS for tumors (Miltenyi tumor dissociation kit 130-096-730, C-tubes, 130-093-237) and homogenized using an Miltenyi gentleMACS™ Octo Dissociator with Heaters (130-096-427). Cells were pelleted at 800×g for 5 min at 4° C. and re-suspended in 5 mL of PBS+2% FBS+1 mM EDTA (PFB) and overlaid onto 5 mL of Lympholyte-M (Cedarlane). Lympholyte columns were spun in centrifuge at 1500×g for 20 min at room temperature with no brake. Lymphocyte layer was washed with PBF. Cell pellets were gently re-suspended in 500 μL of PFB with Fc block (BD Biosciences 553142). In 96-well plate, cells were mixed with a solution of SIINFEKL teramer (MBL), representing the immunodominant antigen in B160VA tumors, according to the manufacturers instruction and incubated for 10 minutes at room temperature. Antibody staining cocktails containing the following: Anti-CD45-AF488 (Biolegend 100723), anti-CD3-BV785 (Biolegend 100232), Anti-CD4-PE (eBioscience 12-0041), anti-CD8a-APC (eBioscience 17-0081), anti-CD44-APC-Cy7 (Biolegend 103028), anti-CD19-BV711 (Biolegend 11555), anti-CD127 (135010), anti-KLRG1 (138419), were added and incubated at room temperature for 30 minutes. Cells were washed 3 times with PFB. Cells were fixed in PFB with 1% paraformaldehyte for 1 minutes on ice. Cells were washed twice with PFB and stored at 4′C in the dark. Samples were analyzed on an LSR II flow cytometer (Beckman).

Peripheral blood lymphocytes were isolated from treated mice by removal of blood from the tip of the tail (less than 100 μL of blood taken per mouse) followed by sealing of the wound using a cautery pen. Extracted blood is mixed gently with EDTA solution. Red blood cells are lysed using Pharmlyse (BD; Cat# 555899) following manufacturer's protocol. Lymphocytes are pelleted by centrifugation and resuspended in PBS. Cells are stained using Live/Dead Fixable Aqua Dead stain according to manufacturer's protocol (Thermo Fisher, Cat# L34957). Cells are blocked with anti-Fc antisera, and stained with a cocktail containing the following antibodies from Biolegend: anti-CD45 (103116), anti-CD1 lb (117318), anti-CD3 (100228), anti-CD8 (100742), anti-CD127 (135010), anti-KLRG1 (138419), anti-CD19 (115546) and incubated for 30 minutes at 2-8° C. in the dark. Samples are washed and analyzed on a LSFortessa X-20 (BD Biosciences). Short-lived CD8 T cells (SLECs) are defined as live CD3(+)CD8(+)CD19(−)KLRG1(+)CD127(−) events.

TABLE 5
ImmunoPulse ® mIL-12 increased SIINFEKL-tetramer-binding
CD8+ T cells in the spleens of treated, B16OVA tumor-bearing
mice. Mice were electroporated intratumorally once on Day 0
using 350 V/cm, 10-msec pulses, 300 ms pulse frequency,
with 0.5 cm acupuncture needles.

| | Percent of CD3+ CD8+ CD44+ |
|---|---|
| | T cells that are SIINFEKL-tetramer |
| Treatment | positive on Day 13, n = 6 |
| ImmunoPulse ® mIL-12 | 2.36 +/− 0.75 |
| ImmunoPulse ® pUMVC3 | 0.24 +/− 0.04 |
| Untreated | 0.10 +/− 0.04 |

ImmunoPulse® mIL-12 induces an increase in circulating CD8(+) T cells directed against the SIINFEKL peptide from ovalbumin, the dominant antigen in B160VA tumors. These data indicate that local IL-12 therapy can lead to system tumor immunity in mice.

TABLE 6
ImmunoPulse ® mIL-12 alters the immune
environment in B16OVA contralateral
(untreated) tumors. Mice were electroporated
intratumorally once on Day 0 using 350 V/cm,
10-msec pulses, 300 ms pulse frequency, with 0.5 cm
acupuncture needles. The composition of
infiltrating lymphocytes (TIL) in untreated tumors
measured 18 days after treatment is shown.

| | Composition of TIL in untreated tumors | | |
|---|---|---|---|
| | Mean +/− SEM, n = 6 | | |
| | % CD3 + CD8 + | % SLEC T | CD8/Treg |
| Treatment | T cells % | cells | T cell ratio |
| ImmunoPulse ® mIL-12 | 14.8 +/− 2.7 | 1.0 +/− 0.1 | 1892 +/− 602 |
| ImmunoPulse ® | 3.6 +/− 1.1 | 0.2 +/− 0.07 | 659 +/− 129 |
| pUMVC3 control | | | |
| Untreated | 2.9 +/− 0.9 | 0.09 +/− 0.03 | 753 +/− 288 |

TABLE 7
Combination treatment with ImmunoPulse ® mIL-12
and Anti-PD-L1 induces detectable
levels of short-lived effector T cells (SLEC) in
the blood of treated mice. ImmunoPulse ® mIL-12
was performed with 350 V/cm, 10-msec pulses
on Day 0 and Day 7 (8 and 15 days after tumor
cell implantation). Anti-PD-Ll antibody was
injected IP twice weekly starting on Day 7. Blood
was drawn and analyzed on Day 17.

| | Percentage of circulating |
|---|---|
| | T cells that are |
| | SLECs in PBMCs |
| Treatment | Mean +/− SEM, n = 5 |
| ImmunoPulse ® mIL-12 + IgG control | 6.7 +/− 1.7 |
| ImmunoPulse ® mIL-12 + Anti-PD-L1 | 24.2 +/− 3.5 |

ImmunoPulse® mIL-12 treatment of B16 tumor-bearing mice induced an increase in SLEC T cells in untreated tumors. When administered in combination with antagonistic PD-Ll antibodies, a significant increase in SLECs was also detected in peripheral blood samples in tumor-bearing mice. These results suggest a robust systemic tumor immunity induced by these therapies, corroborating the reduced growth of untreated, contralateral tumors (Tables 3 and 4).

### E. NanoString® Analysis of Mouse Gene Expression

NanoString was used for analysis of changes in gene expression in untreated tumors induced by ImmunoPulse® mIL-12. Tumor tissue was carefully harvested from mice using scalpel and flash frozen in liquid nitrogen. Tissues were weighed using a balance (Mettler Toledo, Model ML54). 1 ml of Trizol (Thermo Fisher Scientific, Waltham, Mass.) was added to the tissue and homogenized using a probe homogenizer on ice. RNA was extracted from Trizol using manufacturer's instructions. Contaminating DNA was removed by DNase (Thermo Fisher, Cat no: EN0525) treatment. Total RNA concentrations were determined using the NanoDrop ND-1000 spectrophotometer (Thermo Fisher Scientific). Gene expression profiling was performed using NanoString® technology. In brief, 50 ng of Total RNA was hybridized at 96° C. overnight with the nCounter® (Mouse immune ‘v1’ Expression Panel NanoString® Technologies). This panel profilesh 561 immunology-related mouse gene as well as two types of built-in controls: positive controls (spiked RNA at various concentrations to evaluate the overall assay performance) and 15 negative controls (to normalize for differences in total RNA input). Hybridized samples were then digitally analyzed for frequency of each RNA species using the nCounter SPRINT® profiler. Raw mRNA abundance frequencies were analyzed using the nSolver° analysis software 2.5 pack. In this process, normalization factors derived from the geometric mean of housekeeping genes, mean of negative controls and geometric mean of positive controls were used.

TABLE 8
ImmunoPulse ® mIL-12 caused an increase in intratumoral levels of lymphocyte and
monocyte cell surface markers in both primary and contralateral tumors. Fold change of treated
vs. untreated mice values are shown.

| Immune | ImmunoPulse mIL-12 | | ImmunoPulse pUMVC3 | | Untreated | |
|---|---|---|---|---|---|---|
| Checkpoint | Mean +/− SEM n = 5 | | Mean +/−SEM n = 4 | | Mean +/− SEM n = 3 | |
| Protein RNA | Primary | Contralateral | Primary | Contralateral | Primary | Contralateral |
| CD45 | 11.54 +/− 1.65 | 3.55 +/− 0.40 | 1.70 +/− 0.72 | 1.26 +/− 0.51 | 1.00 +/− 0.38 | 1.00 +/− 0.50 |
| CD3 | 13.16 +/− 2.95 | 5.30 +/− 0.72 | 1.26 +/− 0.38 | 1.09 +/− 0.32 | 1.00 +/− 0.22 | 1.00 +/− 0.40 |
| CD4 | 2.35 +/− 0.39 | 2.74 +/− 0.44 | 0.73 +/− 0.18 | 1.00 +/− 0.22 | 1.00 +/− 0.20 | 1.00 +/− 0.09 |
| CD8 | 16.28 +/− 3.10 | 4.60 +/− 0.50 | 1.23 +/− 0.32 | 1.00 +/− 0.15 | 1.00 +/− 0.14 | 1.00 +/− 0.45 |
| KLRC1 | 14.03 +/− 2.73 | 5.62 +/− 0.23 | 1.16 +/− 0.45 | 1.28 +/− 0.44 | 1.00 +/− 0.07 | 1.00 +/− 0.43 |
| KLRD1 | 4.64 +/− 1.00 | 4.17 +/− 0.33 | 1.05 +/− 0.27 | 1.65 +/− 0.45 | 1.00 +/− 0.20 | 1.00 +/− 0.30 |
| CD11b | 11.13 +/− 2.39 | 4.17 +/− 0.48 | 1.55 +/− 0.52 | 1.11 +/− 0.40 | 1.00 +/− 0.42 | 1.00 +/− 0.34 |

TABLE 9
ImmunoPulse ® mIL-12 caused an increase in intratumoral levels of INF-γ
regulated genes in both primary and contralateral tumors. Fold change of
treated vs. untreated mice values are shown

| | ImmunoPulse mIL-12 | | ImmunoPulse pUMVC3 | | Untreated | |
|---|---|---|---|---|---|---|
| IFN-γ related | Mean +/− SEM n = 5 | | Mean +/− SEM n = 4 | | Mean +/− SEM n = 3 | |
| RNA | Primary | Contralateral | Primary | Contralateral | Primary | Contralateral |
| IFNγ | 8.63 +/− | 1.80 +/− | 0.76 +/− | 0.98 +/− | 1.00 +/− | 1.00 +/− |
| | 1.38 | 0.44 | 0.22 | 0.43 | 0.15 | 0.29 |
| CD274 | 12.47 +/− | 7.03 +/− | 1.00 +/− | 1.18 +/− | 1.00 +/− | 1.00 +/− |
| (PD-L1) | 2.24 | 2.30 | 0.30 | 0.83 | 0.48 | 0.84 |
| CXCL10 | 3.18 +/− | 2.26 +/− | 0.99 +/− | 1.44 +/− | 1.00 +/− | 1.00 +/− |
| | 0.58 | 0.42 | 0.30 | 0.85 | 0.43 | 0.73 |
| CXCL11 | 5.02 +/− | 3.14 +/− | 0.74 +/− | 1.38 +/− | 1.00 +/− | 1.00 +/− |
| | 0.74 | 0.41 | 0.10 | 0.82 | 0.16 | 0.55 |
| CXCL9 | 5.92 +/− | 3.75 +/− | 1.03 +/− | 1.67 +/− | 1.00 +/− | 1.00 +/− |
| | 0.60 | 0.57 | 0.31 | 1.37 | 0.50 | 0.85 |
| H2A-a | 9.21 +/− | 6.63 +/− | 1.26 +/− | 1.52 +/− | 1.00 +/− | 1.00 +/− |
| | 1.86 | 2.21 | 0.36 | 0.99 | 0.61 | 1.28 |
| H2k-1 | 4.23 +/− | 3.71 +/− | 1.06 +/− | 1.42 +/− | 1.00 +/− | 1.00 +/− |
| | 1.02 | 0.68 | 0.19 | 0.52 | 0.54 | 0.87 |
| IRF 1 | 4.18 +/− | 2.72 +/− | 1.09 +/− | 1.28 +/− | 1.00 +/− | 1.00 +/− |
| | 0.28 | 0.46 | 0.28 | 0.93 | 0.45 | 0.78 |
| PDCD1 | 3.80 +/− | 2.78 +/− | 1.13 +/− | 1.18 +/− | 1.00 +/− | 1.00 +/− |
| (PD-1) | 0.48 | 0.84 | 0.25 | 0.37 | 0.28 | 0.56 |
| Stat 1 | 3.51 +/− | 3.47 +/− | 1.04 +/− | 1.36 +/− | 1.00 +/− | 1.00 +/− |
| | 0.28 | 0.68 | 0.26 | 0.79 | 0.48 | 0.79 |
| TAP 1 | 3.80 +/− | 2.84 +/− | 1.17 +/− | 1.36 +/− | 1.00 +/− | 1.00 +/− |
| | 0.48 | 0.37 | 0.27 | 0.85 | 0.50 | 0.97 |
| CCL5 | 24.47 +/− | 14.59 +/− | 2.21 +/− | 1.48 +/− | 1.00 +/− | 1.00 +/− |
| | 7.81 | 2.97 | 0.72 | 0.40 | 0.29 | 0.40 |
| CCR5 | 11.29 +/− | 3.70 +/− | 1.31 +/− | 1.21 +/− | 1.00 +/− | 1.00 +/− |
| | 2.72 | 0.70 | 0.42 | 0.42 | 0.27 | 0.40 |
| GZMA | 11.08 +/− | 4.60 +/− | 1.43 +/− | 2.05 +/− | 1.00 +/− | 1.00 +/− |
| | 1.18 | 0.96 | 0.53 | 0.91 | 0.23 | 0.22 |
| GZMB | 3.11 +/− | 2.11 +/− | 0.68 +/− | 1.47 +/− | 1.00 +/− | 1.00 +/− |
| | 0.83 | 0.10 | 0.22 | 0.67 | 0.33 | 0.47 |
| PRF1 | 8.21 +/− | 2.06 +/− | 1.0 +/− | 1.13 +/− | 1.00 +/− | 1.00 +/− |
| | 2.27 | 0.26 | 0.32 | 0.45 | 0.23 | 0.39 |

Gene expression analysis of tissue from treated and untreated tumors corroborate flow cytometric analysis showing a robust increase in tumor TIL. In addition, an increase in interferon gamma-regulated genes suggest induction of an immunostimulatory environment within the tumors. A significant increase in expression of checkpoint proteins indicate that ImmunoPulse® mIL-12 can increase the substrate for the action of checkpoint inhibitors used in combination.

### IL Clinical Trial Data

### A. Rationale

Inhibition of the PD-L1/PD-1 axis with monoclonal antibodies (mAbs) as a monotherapy demonstrates objective response rates (ORR) in the range of 20-40% (i.e. Chen et al., 2015, J. Clin. Invest. 125:3384). Unfortunately, even in melanoma, which is considered to be one of the most immunoresponsive types of solid tumor, the majority of patients will not respond to monotherapy with anti-PD-1 agents. These primary PD-1-nonresponders represent a significant unmet medical need that may benefit from a combination therapy tailored to convert a large portion of this cohort into a PD-1-responder population.

PD-1 was shown to be expressed on activated lymphocytes including peripheral CD4+ and CD8+ T-cells, B cells, T regs and Natural Killer (NK) cells (Agata et al., 1996, Int. Immunol 8:765; Vibhakar et al., 1997, Exp. Cell Res. 232:25). Expression has also been shown during thymic development on CD4-CD8- (double negative) T-cells as well as subsets of macrophages and dendritic cells (Nishimura, 2000, J. Exp. Med. 191:891). The ligands for PD-1 (PDL-1 and PD-L2) are constitutively expressed or can be induced in a variety of cell types and various tumors (Francisco et al., 2010, Immunol. Rev. 236:219). Patients with a high density of CD8+PD-1+ TIL, usually seen in distinct clusters in association with PD-L1+ cells, often have a high probability of response to anti-PD-1 monotherapy. These patients benefit from a strong endogenous antitumor response, leading to the generation of cytotoxic T lymphocytes. On the other hand, the absence of significant numbers of TILs in melanoma is highly correlated with the lack of response to PD-1 therapy (Tumeh et al., 2014, Nature 515:568). Combining the anti-PD-1 agent, pembrolizumab, with an agent capable of driving an effective T cell response, such as IL-12, may increase the immunogenicity in the non-responder phenotype and enhance response to PD-1/PD-L1 blockade.

Intratumoral injection of a plasmid expressing IL-12 (e.g., tavo) delivered by electroporation (e.g., ImmunoPulse® IL-12) demonstrated reduced tumor volume along with an increase in intratumoral infiltrates of CD4+ and CD8+ in the poorly immunogenic and anti-PD1 refractory B16.F10 mouse melanoma model (see above). The dose proportional increase in IL-12 protein expression and tumor levels of IFN-γ seen in the Phase I trial further demonstrate intratumoral changes post-treatment (Daud et al., 2008, J. Clin Onco1.26:5896). Emerging Phase II data indicate a doubling of intratumoral NK cells from pre-treatment through day 11 and at day 39, and increased frequency in activated circulating NK cells (OncoSec Medical, 2013 press release).

### B. Protocol

An ongoing multi-center, open-label, single arm trial, with 23 patients enrolled is assessing the best overall response rates to treatment with the combination of tavokinogene teslaplasmid (tavo) delivered by electroporation (Intratumoral ImmunoPulse® IL-12) and pembrolizumab IV, in a pre-selected patient population that would be expected to have very low response rates to pembrolizumab monotherapy based on published biomarkers (Loo and Daud, 2017, J Clin Invest Insight 2:e93433; Daud et al., 2016, J Clin Invest 126:3447)

Tissue biopsies of all patients were collected prior to enrollment to assess for study eligibility. Patients were selected based on a flow cytometric assay, quantifying the frequency of intratumoral CD8⁺ T cells that are PD-1^{hi}CTLA4^{hi} partially exhausted cytotoxic lymphocytes (referred to as peCTL; Loo and Daud, 2017, J Clin Invest Insight 2:e93433; Daud et al., 2016, J Clin Invest 126:3447). The purpose of this patient pre-selection was to enrich the study population for patients that would be unlikely to respond to anti-PD-1/PD-L-1 monotherapy. Each pembrolizumab treatment cycle was 3 weeks. Patients initiated treatment of pembrolizumab concurrently with the first cycle of intratumoral ImmunoPulse® IL-12.

TABLE 10
Treatment Regimen

| | | | | |
|---|---|---|---|---|
| Pembrolizumab | 200 mg | Intravenous | Day 1 of each | Each cycle is 3 |
| (anti-PD-1) | | | cycle | weeks (21 days) |
| ImmunoPulse ® IL-12 | ¼ tumor volume at | Intratumoral | Days 1, 5 , 8 of | |
| | concentration of 0.5 | | each odd cycle | |
| | mg/mL tavo¹ | | | |

All trial treatments were administered on an outpatient basis.

Pembrolizumab was administered at 200 mg once per treatment cycle (i.e., every 3 weeks). Pembrolizumab was administered on day 1 of each cycle (±2 days) after all procedures/assessments have been completed. Pembrolizumab was administered as a 30 minute IV infusion (treatment cycle intervals may be increased due to toxicity). Target infusion time was 30 minutes: −5 min/+10 min).

ImmunoPulse® IL-12 was administered at each odd cycle as long as the subject has at least one accessible superficial lesion (ASL) for treatment. An ASL was defined as meeting the following criteria; (1) at least 0.3 cm×0.3 cm in longest perpendicular diameters, (2) in a suitable location for application of electroporation. In a case where a subject had multiple ASLs, the maximum number of lesions were treated at each cycle, keeping in mind, (1) patient tolerability, and (2) not to exceed the maximum daily dose of 20 mL. Prior to initiation of a new treatment cycle of pIL-12 EP, the investigator determined ASLs for treatment during that cycle. The same ASLs were treated on each day of the cycle (i.e. Days 1, 5, 8). Previously treated, previously identified lesions present at baseline that were left untreated, and/or new lesions which appear during the course of the study that meet the definition of an ASL may be treated as long as the maximum plasmid injection volume per patient per day did not exceed 20 mL. If no ASLs are present at subsequent cycles, the subject may skip that cycle of ImmunoPulse® IL-12 and continue on the study calendar.

### C. Electroporation (EP) Procedure

DNA plasmid vector, tavo, contains the human IL-12 p35 and p40 subunits that are separated by an internal ribosomal entry site and are driven by a single CMV promoter. A schematic drawing of this plasmid structure is shown in FIG. 1.

Prior to plasmid injection, local anesthesia was administered by various methods (e.g. ice or 1% lidocaine injected around the lesion). In addition, the patient may be given analgesics or anxiolytics as necessary prior to or during treatment. After injecting the plasmid solution into the accessible tumor, a sterile applicator containing 6 stainless steel electrodes were co-localized around the plasmid injection site that may be into or around the tumor. The applicator was connected to the power supply and six pulses at a field strength (E+) of 1500 V/cm and pulse width of 100 μs at 1-second intervals were administered to each previously injected tumor. EP following intratumoral tavo injection delivers controlled electrical pulses in a square wave pulse pattern, yielding optimal transmembrane potential for electroporation to occur (Hofmann et al.,1999 IEEE Trans Biomed Eng 46:752). The electroporation pulses were between six hexagonal opposing needle electrodes. After the first pulse, the polarity between the opposing needle electrode pairs was reversed and the needle pair was pulsed again. After the initial paired pulse, the pulse delivery was rotated clockwise to the next opposing needle pairs until a total of six pulses were delivered to complete the electroporation sequence. When multiple tumors were being injected on the same day, EP was performed immediately after the plasmid injection for each tumor. Once a tumor has been completely treated, the next tumor can be injected and immediately electroporated.

The OncoSec Medical System (OMS) used to deliver the plasmid consists of two main components: (1) an electrode applicator (e.g. consisting of a reusable handle and disposable needle electrode applicator; “OMS Applicator”) with a sterile disposable applicator tip with needle electrodes (e.g., OMS Applicator Tip) and (2) an electric pulse generation device (e.g., OMS Electroporation Therapy Generator; “OMS Generator”). The OMS Applicator connects to the OMS Generator via a cable with a proximal connector.

### D. Endpoints

The primary endpoint for this trial was to assess the anti-tumor efficacy of the combination of ImmunoPulse® IL-12 and pembrolizumab in patients with low peCTL melanoma using RECIST v1.1. Patients are being evaluated for objective response rates (ORR) approximately every 12 weeks by investigator evaluation and will continue on therapy if they have stable disease or better at the time of disease evaluations, or at the discretion of the principal investigator if they have progressive disease. Therapy was and will continue to be given until disease progression or unacceptable toxicity for up to two years. The only exception will be those patients who experience a confirmed CR; these patients may discontinue treatment at the investigator's discretion. Patients may reinitiate either therapy post-complete remission relapse if the study remains open and the patient meets the conditions outlined in the protocol. Patients are being followed continually for safety and tolerability by assessment of adverse events.

Secondary endpoints include: (1) assessing safety and tolerability of the combination of ImmunoPulse® IL-12 and pembrolizumab (2) assessing duration of response in low peCTL melanoma patients treated with the combination of ImmunoPulse® IL-12 and pembrolizumab (3) assessing progression free survival (PFS) and overall survival (OS) in low T-ex melanoma patients treated with the combination of ImmunoPulse® IL-12 and pembrolizumab (4) assessing the best overall response rate (BORR) determined by immune related- Response Criteria (irRC) or RECIST v1.1.

TABLE 11
Interim Objective Response Rate (ORR) of 22
patients undergoing combination therapy
as measured by RECIST v1.1 at 24 weeks.

| Objective Response by RECIST v1.1 | Number of patients |
|---|---|
| Complete response (CR) | 6 |
| Partial response (PR) | 4 |
| No Response (SD and PD) | 12 |

Patients assessed all had a frequency of PD-1^{hi}CTLA-4^{hi}TIL of <22% (low peCTL status), phenotypes previously associated with a low probability of response to anti-PD-1 (Loo and Daud, 2017, J Clin Invest Insight 2:e93433; Daud et al., 2016, J Clin Invest 126:3447). These patients' age ranged from 39-89. Treatment was well tolerated; 38% of adverse events (AE) were classified as treatment site reactions (grade 1-2) that resolved. One SAE of cellulitis resolved with 5d antibiotics. One grade 3 AE of diarrhea resolved with corticosteroids. The BORR was 48% (9CR, 2PR) based on clinical judgment and/or RECIST v1.1.

Additional exploratory endpoints included investigation of candidate biomarkers, which include PD-L1 expression levels assessed by IHC, and TIL profile assessed by CD8 T cell density in tumor tissue. Changes in other biomarkers and immune responses in tissue and blood were assessed for association with clinical outcome.

### E. Flow Cytometry

Blood samples were obtained from patients for analysis of immune cell subsets by flow cytometry. Peripheral blood mononuclear cells (PBMCs) were isolated from Vacutainer® CPT™ Mononuclear Cell Preparation Tubes (BD Biosciences Franklin Lakes, N.J. cat. #362753), and cryopreserved for batch analysis. Preserved leukocytes were collected from Cyto-Chex® BCT tubes (Streck Omaha, Nebr. cat. #213386).

Frozen PBMCs which were thawed or preserved leukocytes were stained for surface cell markers for 30 minutes at 4′C. Intracellular staining was done using the FoxP3 fix/perm buffer set (Biolegend, Cat# 421403) according to the manufacturer's protocol. Intracellular stains were done for 30 minutes at room temperature.

Ki67 is a protein expressed by dividing cells, and is exclusively expressed by a fraction of PD-1+ CD8 T cells found in tumors and not by T cells in normal tissues or peripheral blood (see, e.g., Ahmadzadeh, et al (2009) Blood 114:1537-1544).

Helper CD4 T cells (CD4 T_{H}) were defined as CD3+CD4+FoxP3⁻; CD8 T cells were defined as CD3+CD4⁻; regulatory T cells (Tregs) were defined as CD3+CD4+FoxP3+CD127⁻; PD-1+CD4 T_{H} cells were defined as CD3+CD4+FoxP3⁻PD-1+; PD-1+Ki67+ CD4 T_{H} cells were defined as CD3+CD4+FoxP3⁻PD-1+Ki67+; PD-1+Ki67⁻ CD4 T_{H} cells were defined as CD3+CD4+FoxP3⁻PD-1+Ki67⁻; PD-1+ CD8 T cells were defined as CD3+CD4⁻PD-1+; PD-1+Ki67+ CD8 T cells were defined as CD3+CD4⁻PD-1+Ki67+; PD-1+Ki67⁻ CD8 T cells were defined as CD3+CD4⁻PD-1+Ki67⁻; natural killer (NK) cells were defined as either CD3⁻CD56^{high}CD16⁻, CD3⁻CD56dimCD16+, CD56^{dim}CD16⁻, or CD3⁻CD56⁻CD16+. Proliferating Effector Memory T cells were defined as CD3+CD8+CCR7⁻CD45RA⁻Ki67+. Short-lived effector T cells (SLECS) were defined as CD3+CD8+CCR7⁻CD45RA+KLRG1+.

TABLE 12
Increase in proliferating, effector memory T cells
in patients' blood were observed after
one cycle of treatment correlated with clinical response.
Responders were defined as patients with
complete response (CR) or Partial response (PR);
non-responders were defined as having stable
disease (SD) or progressive disease (PD).

| | Percent of CD8⁺CCR7⁻CD45RA⁻ | |
|---|---|---|
| | T cells that were Ki67⁺ | |
| Patient population | Screen (mean +/− SD) | Cycle 2, Day 1 (mean +/− SD) |
| Responders n = 5 | 3.00 +/− 1.44 | 6.91 +/− 2.57 |
| Non-responders n = 4 | 1.96 +/− 1.22 | 2.58 +/− 0.89 |

TABLE 13
Increase in Short-Lived Effector Cells (SLEC)
in patients' blood at cycle 2, Day 1
(C2D1) or treatment correlated with clinical response.
Responders were defined as patients with
complete response (CR) or Partial response (PR);
non-responders were defined as having stable
disease (SD) or progressive disease (PD)

| | Ratio C2D1/screen value for % CD8 + CCR7⁻ |
|---|---|
| Patient population | CD45RA + KLRG1 + |
| Responders n = 4 | 1.29 +/− 0.24 |
| Non-responders n = 4 | 0.78 +/− 0.26 |

Analysis of patients' blood samples after one cycle of treatment indicated that responding patients had a greater increase in the percentage of PBMCs that were proliferating effector memory T cells (1.8 fold higher) and short-lived effector cells (1.7 fold higher) than did non-responding patients. Patient response was measured by RESIST at week 24 (end of cycle 8 of treatment).

### F. NanoString Analysis of Relative Gene Expression in Patient Tumor Biopsies

Tumor tissue samples procured by punch biopsy or fine needle aspirate (FNA) were homogenized in phosphate-buffered saline without Ca²+ or Mg²⁺ (Thermo Fisher Scientific, Carlsbad, Calif.) with protease inhibitors (cComplete-mini, EDTA-free; Roche Life Science, Indianapolis, Ind.). Clarified supernatants and cell pellets were stored separately at −80° C. Total RNA was isolated from cell pellets using -RNeasy FFPE kit (Qiagen, Hilden, Germany) according to the manufacturer's protocol. Total RNA concentrations were determined using the NanoDrop ND-1000 spectrophotometer (Thermo Fisher Scientific) and quality was assessed using the 2100 Bioanalyzer (Agilent, Santa Clara, Calif.) for both the standard and smear analyses by LabCorp (Seattle, Wwash.). Samples were excluded from analysis if Bioanalyzer RIN score was less than 2.0 and smear analysis indicated <30% of RNA were less than 300 bp. Total RNA was used with the NanoString® nCounter system, according to the manufacturer's protocol (Nanostring® Technologies, Seattle, Wash.) by LabCorp (Seattle, Wash.). In brief, 5 μL (100 ng) of total RNA was hybridized at 96° C. overnight with the nCounter® (Human Immunology v2 Gene Expression Panel, NanoString® Technologies). This panel profiles 594 immunology-related human genes as well as two types of built-in controls: positive controls (spiked RNA at various concentrations to evaluate the overall assay performance) and 15 negative controls (to normalize for differences in total RNA input). In addition, the human Pan-Cancer IO 360 Beta which profiles 770 genes from 13 cancer-associated canonical pathways is run on the same biopsies. Hybridized samples were then digitally analyzed for frequency of each RNA species using the nCounter SPRINT™ profiler. Raw mRNA abundance frequencies were analyzed using the nSolver® analysis software 3.0 pack. In this process, normalization factors derived from the geometric mean of housekeeping genes, mean of negative controls and geometric mean of positive controls were used. Gene expression analysis was assessed as the ratio of the paired IL-12-treated and the screening pre-treatment patient biopsies for subsets of immune-related genes. (GraphPad Prism, La Jolla, Calif.). Responder (R) cohorts were defined as patients with a partial or complete response as measured by RECISTv1.1.

TABLE 14
The combination of ImmunoPulse ® IL-12
and pembrolizumab treatment increased
expression of gene that comprise an INFγ
signature gene set (Ribas A et al., 2015, J Clin Oncol.,
33:Abstr 3001; Ayers M et al., 2015,
J Immunother Cancer., 3:80) in treated lesions on cycle 2,
day 1 (C2D1) as compared to screening biopsies.

| | Log2 Fold change in expression: | |
|---|---|---|
| | C2D1 vs. Screening biopsy | |
| | Mean +/− SEM | |
| INFγ related genes | Responders n = 8 | Non-responders n = 8 |
| CXCL10 | 1.57 +/− 0.93 | 0.08 +/− 0.75 |
| CXCL9 | 2.00 +/− 1.26 | 0.10 +/− 0.76 |
| HLA-DRA | 1.73 +/− 0.34 | 0.05 +/− 0.39 |
| IDO1 | 2.51 +/− 0.87 | −0.31 +/− 0.67 |
| IFN □ | 2.43 +/− 0.57 | 0.76 +/− 0.65 |
| STAT1 | 0.85 +/− 0.64 | 0.36 +/− 0.51 |

TABLE 15
The combination of ImmunoPulseR IL-12
and pembrolizumab treatment increased
expression of genes present in activated
Natural Killer (NK) cells in treated lesions on cycle 2, day 1
(C2D1) as compared to screening biopsies.

| | Log2 Fold change in expression: C2D1 vs. | |
|---|---|---|
| | Screening biopsy | |
| Genes for natural killer | Mean +/− SEM | |
| (NK) cell markers | Responders n = 8 | Non-responders n = 8 |
| KLRC1 | 2.09 +/− 0.36 | 0.11 +/− 0.44 |
| KLRC2 | 2.02 +/− 0.43 | −0.10 +/− 0.48 |
| KLRB1 | 2.75 +/− 0.56 | 1.00 +/− 0.55 |
| KLRG1 | 1.52 +/− 0.23 | 0.59 +/− 0.29 |
| KLRG2 | 0.80 +/− 0.75 | −0.70 +/− 0.40 |

TABLE 16
The combination of ImmunoPulse ® IL-12 and
pembrolizumab treatment increased
expression of genes that function in antigen
presentation in treated lesions on cycle 2, day 1 (C2D1)
as compared to screening biopsies

| | Log2 Fold change in expression: | |
|---|---|---|
| | C2D1 vs. Screening biopsy | |
| Genes associated with | Mean +/− SEM | |
| antigen presentation | Responders n = 8 | Non-responders n = 8 |
| CIITA | 2.50 +/− 0.72 | 0.37 +/− 0.46 |
| LILRA4 | 1.56 +/− 0.64 | −0.37 +/− 0.73 |

TABLE 17
The combination of ImmunoPulse ® IL-12 and
pembrolizumab treatment increased
expression of genes that function in T cell survival
and T cell mediated cytotoxicity in treated
lesions on cycle 2, day 1 (C2D1) as compared
to screening biopsies

| | Log2 Fold change in expression: | |
|---|---|---|
| | C2D1 vs. Screening biopsy | |
| T cell cytotoxicity and | Mean +/− SEM | |
| survival related genes | Responders n = 8 | Non-responders n = 8 |
| Granzyme B | 3.22 +/− 0.64 | 0.46 +/− 0.62 |
| Granzyme K | 2.72 +/− 0.60 | 0.52 +/− 0.42 |
| TNF □ | 1.52 +/− 0.35 | 0.17 +/− 0.32 |
| IL-7 receptor | 1.83 +/− 0.27 | 0.27 +/− 0.53 |
| IL-2 receptor □ | 2.02 +/− 0.48 | 0.09 +/− 0.44 |

TABLE 18
The combination of ImmunoPulse ® IL-12
and pembrolizumab treatment increased
expression of immune checkpoint genes in treated
lesions on cycle 2, day 1 (C2D1) as compared
to screening biopsies

| Immune | Log2 Fold change in expression: | |
|---|---|---|
| checkpoint | C2D1 vs. Screening biopsy | |
| protein | Mean +/− SEM | |
| gene | Responders n = 8 | Non-responders n = 8 |
| CD274 (PD-L1) | 2.53 +/− 0.61 | 0.19 +/− 0.42 |
| PDCD1 (PD-1) | 2.39 +/− 0.60 | 0.08 +/− 0.55 |
| PDCD1LG2 (PD-L2) | 1.65 +/− 0.39 | 0.38 +/− 0.39 |
| CTLA4 (transmembrane) | 1.58 +/− 0.57 | 0.34 +/− 0.46 |
| CTLA4 (total) | 1.95 +/− 0.43 | 0.18 +/− 0.39 |
| LAG3 | 2.84 +/− 0.90 | 0.77 +/− 0.50 |

TABLE 19
The combination of ImmunoPulse ® IL-12
and pembrolizumab treatment increased
expression of lymphocyte cell surface markers
in treated lesions on cycle 2, day 1 (C2D1) as
compared to screening biopsies.

| Lymphocyte | Log2 Fold change in expression: C2D1 vs. Screening | |
|---|---|---|
| cell surface | biopsy Mean +/− SEM | |
| marker | Responders n = 8 | Non-responders n = 8 |
| CD8b | 2.01 +/− 0.46 | 0.04 +/− 0.55 |
| CD3D | 2.65 +/− 0.67 | 0.45 +/− 0.51 |
| CD3E | 2.60 +/− 0.62 | 0.25 +/− 0.41 |
| CD3zeta (CD247) | 2.28 +/− 0.50 | 0.25 +/− 0.42 |
| CXCR6 | 2.46 +/− 0.43 | 0.52 +/− 0.44 |
| CXCL13 | 3.29 +/− 1.31 | 0.13 +/− 1.11 |
| IL2 receptor gamma | 1.87 +/− 0.49 | 0.22 +/− 0.47 |

NanoString® analysis of patient biopsies shows an upregulation of productive immune-related genes in tumors suggesting that treatment can alter the tumor microenvironment to recruit and activate T and NK cells, enable antigen presentation, and increase the presentation of immune checkpoint proteins as substrate for checkpoint inhibitors. The increase in expression of all the genes shown in Table 4, 5, 6, 7, 8 and 9 was significantly higher in responding patients than non-responding patients. The increase in expression of these genes can serve as biomarkers for prediction of overall clinical response.

### G. ELISPot Assay

Blood samples were obtained from patients. Peripheral blood mononuclear cells (PBMCs) were isolated from Vacutainer® CPT™ Mononuclear Cell Preparation Tubes (BD Biosciences Franklin Lakes, NJ cat. #362753) and cryopreserved for batch analysis.

PBMCs were thawed and rested overnight at 37° C. The cells were plated in triplicates of 1.0×10⁵ cells each and incubated with gp100, NY-ESO-1, Mage-A3, Melan-A/MART-1 peptides (JPT Peptide Technologies Berlin, Germany), leucoagglutinin PHA-L (Sigma-Aldrich St. Louis, Mo., cat. #L2769) or with no antigen for 48 hours at 37° C. in MultiScreen Filter Plates (EMD Millipore Billerica Massachusetts, cat. #MAIPS4510). Cells secreting IFN-γ were visualized by anti-human-IFN-γ enzyme-linked immunospot assay (ELISpot) (MABTECHNacka Strand Sweden, cat. #3420-2A). Plates were scanned with ELISpot plate reader (Cellular Technology Limited (CTL) Shaker Heights, Ohio-ImmunoSpot Analyzer) and counted using CTL Immunospot 5.0 Analyzer software. Final counts of antigen specific IFN-γ secreting cells were obtained by subtracting the number of spots counted in control wells (no-antigen) from test wells. Samples were accepted for inclusion in the final analysis if the positive control PHA wells had an average >100 spots/well, and negative control (no antigen) wells had <200 spots/well.

TABLE 20
Antigen-specific immune response with INFγ ELISpot.

| | Spots/100,000 cells (mean +/− SD) | | | |
|---|---|---|---|---|
| Patient sample | Unstimulated | gp100 | Trp2 | PHA |
| Healthy donor | 6.25 +/− 7.42 | 4.00 +/− 0.00 | 9.25 +/− 2.48 | 714.3 +/− 114.9 |
| Responder, screen | 0.00 +/− 0.00 | 23.00 +/− 7.07 | 195.3 +/− 50.56 | 729.8 +/− 89.45 |
| Responder, C2D1 | 0.50 +/− 0.00 | 28.25 +/− 8.84 | 293.0 +/− 97.58 | 784.8 +/− 57.63 |
| Non-Responder, screen | 4.00 +/− 0.71 | 2.75 +/− 1.77 | 8.25 +/− 0.35 | 995.3 +/− 154.5 |
| Non-responder, C2D1 | 15.75 +/− 6.72 | 15.50 +/− 3.54 | 23.00 +/− 7.78 | 1029 +/− 165.5 |

ELISpot measurement of the ex-vivo production of IFN-γ from lymphocytes taken from patients' blood demonstrate that compared to the healthy donor or non-responding patient, the patient responding to the therapy had a measurable response to the melanoma-specific antigens gp110 and Trp2 at screening with an increase in spots/100,000 cells after one cycle of treatment.

### H. Immunohistochemistry Analysis of Patient Biopsies: Chromagenic IHC Assay

Formalin-fixed paraffin-embedded (FFPE) biopsy tissues were sectioned at a thickness of approximately 5μm onto positively-charged slides.

Slides were stained and scored in accordance to the FDA-approved instructions provided in the Dako PD-L1 IHC 22C3 pharmDx kit documentation (PhenoPath). Percent tumor cell positivity in the overall tumor was reported to the nearest decile, and positivity at the tumor-stroma margin was noted when greater than 50%. For CD8 marker staining (DAKO Cat# M7103), results are presented as percentage of cells, in both intratumoral and peritumoral areas, to the nearest decile. H&E-stained slides were also examined for assessment of overall morphology

PD-L1 and CD8 chromogenic analysis of biopsies from patients taken at screening were compared to one taken after the first cycle of treatment, on cycle 2, day 1. Responder (R) cohorts were defined as patients with a partial or complete response as measured by RECISTv1.1. Non-responder (NR) cohorts were defined as patients with progression or stable disease as measured by RECISTv1.1.

TABLE 21
Changes in PD-L1 and CD8 protein levels in
patient biopsies from responders (R) and
non-responder (NR) cohorts on cycle 2, day 1
of treatment as compared to screening biopsies.

| | Percent tumor positive signal: Mean +/− SEM | | | |
|---|---|---|---|---|
| | Responders | | Non-responders | |
| Protein | Screening n = 4 | C2D1 n = 2 | Screening n = 6 | C2D1 n = 5 |
| PD-L1 | 10.0 +/− 5.8 | 65.0 +/− 5.0 | 7.5 +/− 3.1 | 26.0 +/− 14.7 |
| CD8 | 18.0 +/− 10.6 | 40.0 +/− 10.0 | 19.2 +/− 8.4 | 26.0 +/− 8.7 |

### I. Immunohistochemistry Analysis of Patient Biopsies: Multispectral IHC Assay

Tissue sections were cut at 5 μm from formalin-fixed paraffin-embedded blocks. All the sections were deparaffinized and subjected to heat-induced epitope retrieval in citrate buffer pH 9.0 (Biogenex, Fremont, Calif.). 6-plex panel immunohistochemistry was performed for each tissue slide using the following antibodies: anti-FoxP3 (clone 236A/E7, Abcam, Cambridge, Mass.), anti-PD-L1 (clone E1L3N, Cell Signaling, Danvers, Mass.), anti-CD8 (clone SP16, Spring Bioscience, Pleasanton, Calif.), anti-CD3 (clone SP7, Spring Bioscience), anti-CD163 (clone MRQ26, Ventana, Tucson, Ariz.), anti-Cytokeratin (clone AE1/AE3, DAKO, Carpinteria, Calif.). Antigen-antibody binding was visualized with TSA-Cy5 (PerkinElmer, Waltham, Mass.), TSA-Cy3 (PerkinElmer), TSA-FITC (PerkinElmer), TSA-Alexa594 (Carlsbad, Calif.), TSA-Cy5.5 (PerkinElmer), and TSA-Coumarin (PerkinElmer) respectively. Microwave treatment in citrate buffer pH 6.0 was performed between antibodies detection to prevent cross-reactivity. Tissue slides were incubated with DAPI as counterstain and coverslipped with VectaShield mounting media (Vector Labs, Burlingame, Calif.).

Digital images were captured with PerkinElmer Vectra platform. Tumor areas with the highest immune cell (CD3+CD8+) infiltrates were scanned at 20× and selected for analysis. Three images of 0.36 mm² each were analyzed per sample with InForm Software (PerkinElmer). The total number of cells were enumerated for the following phenotypes: PD-L1+ tumor cells+, PD-L1+ other cells, CD3+PD-L1+, CD3+PD-L1− FoxP3+, CD8+ PD-L1+, CD8+ PD-L1−, CD163+ PD-L1+, CD163+ PD-L1− in the stroma and tumor compartment.

A hematoxylin and eosin staining was performed for each sample and reviewed by a pathologist to ensure a representative tissue sample. Responder (R) cohorts were defined as patients with a partial or complete response as measured by RECISTv1.1. Non-responder (NR) cohorts were defined as patients with progression or stable disease as measured by RECISTv1.1.

TABLE 22
Changes measured in the ratio of CD8 positive cells to
PD-L1 positive cells (both CD163
positive macrophages and total tumor cells are shown)
in patient biopsies from responders (R) and
non-responder (NR) cohorts on cycle 2, day 1
of treatment as compared to screening biopsies.

| | Log2 Fold Change C2D1: Screening | |
|---|---|---|
| | Mean +/− SEM | |
| Protein marker ratio | Responders n = 6 | Non-responders n = 4 |
| CD8(+):PD-L1(+) | 1.61 +/− 0.87 | −0.08 +/− 0.39 |
| CD8(+):PD-L1(+)CD163(+) | 2.74 +/− 0.75 | 0.06 +/− 0.54 |

The higher frequency of PD-L1 positive cells as well as the increased post-treatment ratio of CD8+:PD-L1+ cells in the responding patients suggests that the combination of ImmunoPulse® IL-12 and pembrolizumab treatment creates an inflamed tumor with the appropriate substrate for the anti-PD-1 checkpoint blockade used in combination.

Two patients with lesions that were not directly treated with ImmunoPulse® IL-12 were analyzed by multispectral IHC and demonstrated evidence of significant lymphocyte infiltration.

The combination ImmunoPulse® IL-12 with pembrolizumab in patients with an anti-PD-1 non-responsive phenotype engendered a 48% clinical response with associated positive immune-based biomarker data and an excellent safety profile. These data suggest that ImmunoPulse® IL-12 modulates the tumor microenvironment to enable an effective anti-PD-1 mAb response in patients otherwise unlikely to respond.

An additional Phase II trial is initiated to test efficacy of ImmunoPulse® IL-12 and Pembrolizumab IV in Patients with Stage III/IV Melanoma that are progressing on anti-PD-1 Antibody monotherapy. Eligible patients are those with pathological diagnosis of unresectable or metastatic melanoma who are progressing or have progressed on pembrolizumab or nivolumab. The study is comprised of a Core study (24 weeks), an Extension Phase and a long-term safety follow-up and assesses if ImmunoPulse® IL-12 in combination with pembrolizumab can convert PD-1 checkpoint inhibitor non-responders to responders.

Core study: Eligible patients are treated with ImmunoPulse® IL-12 at accessible lesions on Days 1, 5 and 8 every 6 weeks and with IV pembrolizumab (200 mg) on Day 1 of each 3-week cycle for 24 weeks. As many accessible lesions are treated, as deemed feasible by the treating physician at each visit.

Patients who complete 24 weeks of treatment (Core study), at the investigators discretion, enter an Extension phase and continue to receive the combined treatment of ImmunoPulse® IL-12 and pembrolizumab for up to 35 cycles of pembrolizumab from baseline (approximately 2 years) or until subsequent disease progression.

What is claimed is:

1. A method of treating cancer in a subject, the method comprising:
(a) measuring a level of expression of one or more genes selected from the group consisting of: CXCL9, CXCL10, HLA-DRA, IDOL IFNγ, STAT1, KLRC1, KLRC2, KLRB1, KLRG1, KLRG2, CIITA, LILRA4, Granzyme B, Granzyme K, TNFα, IL-7 receptor, IL-2 receptor, CD274, PDCD1, PDCD1LG2, CTLA4, LAG3, CD8b, CD3D, CD3E, CD3zeta, CXCR6, and CXCL13 in a tumor sample obtained from the subject after administering a checkpoint inhibitor and IL-12 to the subject, or
(b) measuring a level of proliferating effector memory T cells or short-lived effector T cells in a blood sample obtained from the subject after administering a checkpoint inhibitor and IL-12 to the subject,
wherein an increase in the level of expression of the one or more genes in the tumor sample relative to the level of expression of the one or more genes in a predetermined control or an increase in the level of proliferating effector memory T cells or short-lived effector T cells in the blood sample relative to the level of proliferating effector memory T cells or short-lived effector T cells in a predetermined control indicates the subject is likely to respond to checkpoint inhibitor plus IL-12 combination therapy.

2. The method of claim 1, wherein the checkpoint inhibitor is administered systemically.

3. The method of claim 2, wherein the checkpoint inhibitor comprises an anti-PD-1 or anti-PD-Ll antibody.

4. The method of claim 3, wherein the checkpoint inhibitor comprises: nivolumab, pembrolizumab, pidilizumab, or MPDL3280A.

5. The method of claim 1, wherein the IL-12 is administered by intratumoral electroporation of a nucleic acid encoding IL-12.

6. The method of claim 5, wherein the nucleic acid encoding IL-12 comprises a first nucleic acid sequence encoding an IL-12 p35 subunit and a second nucleic acid sequence encoding an IL-12 p40 subunit wherein the first and second nucleic acid sequences are separated by an internal ribosome entry site (IRES) or a P2A exon skipping motif.

7. The method of claim 1, wherein the checkpoint inhibitor is administered systemically and the IL-12 is administered by intratumoral electroporation of a nucleic acid encoding IL-12.

8. The method of claim 1, wherein measuring the level of expression of the one or more genes in the tumor sample comprises measuring mRNA abundance of the one or more genes.

9. The method of claim 11, wherein the level of proliferating effector memory T cells or short-lived effector T cells is measured by flow cytometry.

10. The method of claim 1, wherein the predetermined control comprises a standard derived from a population of known non-responders to checkpoint inhibitor plus IL-12 combination therapy.

11. The method of claim 1, wherein the subject is a human.

12. A method of treating cancer in a subject comprising:
(a) measuring
(i) an expression level of one or more genes selected from the group consisting of: CXCL9, CXCL10, HLA-DRA, IDO1, IFNγ, STAT1, KLRC1, KLRC2, KLRB1, KLRG1, KLRG2, CIITA, LILRA4, Granzyme B, Granzyme K, TNFα, IL-7 receptor, IL-2 receptor, CD274, PDCD1, PDCD1LG2, CTLA4, LAG3, CD8b, CD3D, CD3E, CD3zeta, CXCR6, and CXCL13 in a tumor sample obtained from the subject, wherein the tumor sample is obtained after administering checkpoint inhibitor and an IL-12 combination therapy to the subject, or
(ii) a level of proliferating effector memory T cells and/or short-lived effector T cells in a blood sample obtained from the subject after administering checkpoint inhibitor and an IL-12 combination therapy to the subject; and
(b) administering a therapeutically effective amount of a checkpoint inhibitor and a therapeutically effective amount of IL-12 to the subject if the expression level of the one or more genes in the tumor sample is increased relative to the expression level of the one or more genes in a predetermined control or if the level of proliferating effector memory T cells and/or short-lived effector T cells in the blood sample is increased relative to the level of proliferating effector memory T cells and/or short-lived effector T cells in a predetermined control.

13. The method of claim 12, wherein the checkpoint inhibitor is administered systemically.

14. The method of claim 13, wherein the checkpoint inhibitor comprises an anti-PD-1 or anti-PD-Ll antibody.

15. The method of claim 14, wherein the checkpoint inhibitor comprises: nivolumab, pembrolizumab, pidilizumab, or MPDL3280A.

16. The method of claim 12, wherein the IL-12 is administered by intratumoral electroporation of a nucleic acid encoding IL-12.

17. The method of claim 16, wherein the nucleic acid encoding IL-12 comprises a first nucleic acid sequence encoding an IL-12 p35 subunit and a second nucleic acid sequence encoding an IL-12 p40 subunit wherein the first and second nucleic acid sequences are separated by an internal ribosome entry site (IRES) or a P2A exon skipping motif.

18. The method of claim 12, wherein the checkpoint inhibitor is administered systemically and the IL-12 is administered by intratumoral electroporation of a nucleic acid encoding IL-12.

19. The method of claim 12, wherein measuring the level of expression of the one or more genes in the tumor sample comprises measuring mRNA abundance of the one or more genes.

20. The method of claim 12, wherein the level of proliferating effector memory T cells and/or short-lived effector T cells is measured by flow cytometry.

21. The method of claim 12, wherein the predetermined control comprises a standard derived from a population of known non-responders to checkpoint inhibitor plus IL-12 combination therapy.

22. The method of claim 12, wherein the subject is a human.
